(19) United States
(12) Patent Application Publication
(10) Pub. No.: US 2022/0022810 A1
(43) Pub. Date: Jan. 27, 2022

(54) Treatment Compliance Tracking by Measuring Symptoms with an Analytical Toilet

(71) Applicant: Medic, Inc., Provo, UT (US)

(72) Inventors: David R. Hall, Provo, UT (US); K. Jeffrey Campbell, Provo, UT (US)

(73) Assignee: Medic, Inc., Provo, UT (US)

(21) Appl. No.: 17/383,969

(22) Filed: Jul. 23, 2021

Related U.S. Application Data
(60) Provisional application No. 63/055,837, filed on Jul. 23, 2020.
(60) Provisional application No. 63/055,673, filed on Jul. 23, 2020.

(51) Int. Cl. A61B 5/00; A61B 5/20; A61B 5/01; A61B 5/11; A61B 5/145; A61B 5/28; G16H 20/10

(52) U.S. Cl. CPC A61B 5/4833; A61B 5/6891; A61B 5/208; A61B 5/0004; A61B 5/0075; A61B 2562/02; A61B 5/1101; A61B 5/14539; A61B 5/28; A61B 5/4875; G16H 20/10; A61B 5/01

(57) ABSTRACT

An analytical toilet comprising a bowl for receiving excreta from a user; a seat for the user; a sensor for detecting a symptom of the user; wherein the sensor transmits data to a processor that uses the data to measure a symptom of the user is disclosed. A method for monitoring a patient comprising providing an analytical toilet comprising: a bowl for receiving excreta from a user; a seat for the user; a sensor for detecting a symptom of the user; having a user use the toilet to deposit excreta; detecting a symptom of the user with the sensor; transmitting data from the sensor to a processor; and using the data to measure the symptom and compare it to the user's normal or expected symptoms is disclosed.

### CROSS-REFERENCE TO RELATED APPLICATIONS

This application claims the benefit under 35 U.S.C. 119(e) of U.S. Provisional Patent Application Nos. 63/055,837 and 63/055,673 both filed on 23 Jul. 2020, which disclosures are incorporated herein by reference in their entireties.

### TECHNICAL FIELD

The present disclosure relates to analytical toilets. More particularly, it relates to analytical toilets equipped to provide health and wellness information to the user.

### BACKGROUND

Patient compliance with taking medicaments outside of a care provider facility is often inconsistent for a variety of reasons from patient forgetfulness to active avoidance due to unpleasant side effects. Patients may not always be truthful about failure to comply with their healthcare providers because of active avoidance or mere embarrassment. There are also difficulties tracking symptoms to measure the effectiveness of a medicament or other treatment, which can vary greatly between even similar patients. A way of tracking treatment compliance and/or effectiveness without requiring frequent expensive clinical visits would be of great benefit.

Similar issues exist in clinical trials of medicaments. Some test participants do not take the medicaments causing the test results to be less reliable and accurate than they otherwise would be. Requiring a participant to use a convenient device that allows tracking of medicament consumption would reduce the cost of clinical trials while increasing the reliability of results.

The ability to track an individual's health and wellness is currently limited due to the lack of available data related to personal health. Many diagnostic tools are based on examination and testing of excreta, but the high cost of frequent doctor's visits and/or scans make these options available only on a very limited and infrequent basis. Thus, they are not widely available to people interested in tracking their own personal wellbeing.

Toilets present a fertile environment for locating a variety of useful sensors to detect, analyze, and track trends for multiple health conditions. Locating sensors in such a location allows for passive observation and tracking on a regular basis of daily visits without the necessity of visiting a medical clinic for collection of samples and data. Monitoring trends over time of health conditions supports continual wellness monitoring and maintenance rather than waiting for symptoms to appear and become severe enough to motivate a person to seek care. At that point, preventative care may be eliminated as an option leaving only more intrusive and potentially less effective curative treatments. An ounce of prevention is worth a pound of cure.

Just a few examples of smart toilets and other bathroom devices can be seen in the following U.S. Patents and Published Applications: U.S. Pat. No. 9,867,513, entitled “Medical Toilet With User Authentication”; U.S. Pat. No. 10,123,784, entitled “In Situ Specimen Collection Receptacle In A Toilet And Being In Communication With A Spectral Analyzer”; U.S. Pat. No. 10,273,674, entitled “Toilet Bowl For Separating Fecal Matter And Urine For Collection And Analysis”; US 2016/0000378, entitled “Human Health Property Monitoring System”; US 2018/0020984, entitled “Method Of Monitoring Health While Using A Toilet”; US 2018/0055488, entitled “Toilet Volatile Organic Compound Analysis System For Urine”; US 2018/0078191, entitled “Medical Toilet For Collecting And Analyzing Multiple Metrics”; US 2018/0140284, entitled “Medical Toilet With User Customized Health Metric Validation System”; and US 2018/0165417, entitled “Bathroom Telemedicine Station.” The disclosures of all these patents and applications are incorporated by reference in their entireties.

A body tremor is an involuntary, rhythmic muscle contraction and relaxation leading to shaking movements in one or more parts of the body. These movements may be motion of one part of the body relative to another. The movements may also manifest as vibrations or waves that propagate through the body similar to an earthquake propagating through the Earth from the earthquake origin. Some tremors can be a physical indication or symptom indicating that a person has a condition for which health or other wellness care should be considered. Additionally, changes to the rhythm of a person's typical tremors can also indicate a change in condition for which health or other wellness care should be considered. Some atypical conditions and circumstances that show correlation with abnormal body tremors include multiple sclerosis, stroke, traumatic brain injury, neurodegenerative diseases that affect parts of the brain (such as Parkinson's disease), use of certain medicines (including particular asthma medication, amphetamines, caffeine, corticosteroids, and some drugs used for psychiatric and neurological disorders), alcohol abuse or withdrawal, mercury poisoning, an overactive thyroid, liver or kidney failure, and anxiety or panic.

There are many ways to classify tremors. One method uses two main categories: resting tremor and action tremor. Resting tremors occur when the muscle(s) is relaxed. Action tremors occur with the voluntary use of a muscle, such as standing still, holding something, walking, and purposefully moving an appendage or other part of the body. Some action tremors are specifically related to goal-oriented, skill related tasks, such as writing or speaking. Another way to classify tremors is based on their appearance, cause, and/or origin. Using this method, there are more than 20 types of tremor. Some common classifications used in this methodology include essential, dystonic, cerebellar, psychogenic, physiologic, enhanced physiologic, Parkinsonian, and orthostatic. A third way to classify tremors is by their frequency or how long it takes for the general motion to repeat. Tremor frequency generally falls within the range of 3-30 Hz or 3-30 times per second. Under 4 Hz may be termed low frequency, 4-7 Hz may be termed medium frequency, and over 7 Hz may be termed high frequency. One method of assessing tremor amplitude uses the following displacement categorizations: (a) no tremor, (b) slight tremor, (c) moderate tremor with displacement under 2 cm, (d) marked tremor with displacement between 2 cm and 4 cm, and (e) severe tremor with displacement over 4 cm.

Notably, physiologic tremors occur in all healthy individuals, are generally not associated with a disease, are generally associated with normal human phenomenon such as heartbeat or maintaining a posture or movement, and may manifest as a fine shaking, such as of the hands and fingers, that is rarely visible to the eye. Neurologic examination results of patients with physiologic tremor are usually normal. Physiologic tremors can be exacerbated, making them significantly more noticeable. An exacerbated physiologic tremor may be a cause for concern and additional health or wellness consideration. Exacerbating factors can include extreme fatigue, stress, intense emotion, low blood sugar (hypoglycemia), an overactive thyroid, medications such as corticosteroids, amphetamines or beta-agonists, heavy metal toxicity, stimulants such as caffeine, fever, and alcohol withdrawal.

Diagnosis of a tremor is based on clinical information obtained from a thorough history and physical examination. A possible first step in the evaluation of a patient with a tremor is to categorize the tremor based on the activation condition, topographic distribution, and frequency that correlate with the manifestation of the tremor. Additional steps can follow if necessary.

Some examples of devices that can detect tremors include U.S. Pat. No. 4,595,023 titled “Apparatus and Method for Detecting Body Vibrations”, U.S. Pat. No. 6,936,016 titled “Method for Analysis of Abnormal Body Tremors”, U.S. Pat. No. 10,064,582 titled “Noninvasive Determination of Cardiac Health and Other Functional States and Trends For Human Physiological Systems”, and JP 6,130,474 with an English translated title of “Weight scale device and pulse wave velocity acquisition method”. The disclosures of all these patents and applications are incorporated by reference in their entireties.

One particular variety of detection, analysis, and trend tracking is related to biomarkers. “A bio-marker, or biological marker is a measurable indicator of some biological state or condition. Biomarkers are often measured and evaluated to examine normal biological processes, pathogenic processes, or pharmacologic responses to a therapeutic intervention. Biomarkers are used in many scientific fields.” (See https://en.wikipedia.org/wiki/Biomarker). Biomarker information can be a valuable resource in providing for the health and wellness of an individual or population. Some of the uses include being used to detect a disease at its earliest stages and monitoring the progression of key health metrics over time.

Initially, the detection of biomarkers was performed on macro-scale samples, but as technology has improved, equipment both takes up a smaller footprint and is able to use smaller and smaller sample sizes. Additionally, as technology improved, smaller and smaller concentrations of individual biomarkers became detectable. Recent innovation has allowed for the creation of circuitry capable of detecting a variety of desirable, specific, individual molecular elements. Current testing of many biomarkers requires the sample to be processed in a laboratory or clinic. Such testing is also costly, inconvenient and time consuming.

One such example is described in U.S. Pat. No. 7,301,199 “Nanoscale Wires and Related Devices”, which outlines the production of nanometer scale circuitry elements. This technology can be used to make nanoscale versions of numerous components. The '199 patent states “for example, semiconductor materials can be doped to form n-type and p-type semiconductor regions for making a variety of devices such as field effect transistors, bipolar transistors, complementary inverters, tunnel diodes, light emitting diodes, sensors, and the like.” (Page 5, Abstract). The disclosure of the '199 patent is incorporated herein by reference in its entirety.

Additionally, US 2018/0088079 “Nanoscale Wires with External Layers for Sensors and Other Applications” disclosed further details related to producing sensors that use nanoscale wires. For example, it teaches “Certain aspects of the invention are generally directed to polymer coating on nanoscale wires that can be used to increase sensitivity to analytes.” (Page 1, Abstract). The disclosure of US 2018/0088079 is incorporated herein by reference in its entirety.

### SUMMARY

In a first aspect, the disclosure provides an analytical toilet comprising a bowl for receiving excreta from a user; a seat for the user; a sensor for detecting a symptom of the user; wherein the sensor transmits data to a processor that uses the data to measure a symptom of the user.

In a second aspect, the disclosure provides a method for monitoring a patient comprising providing an analytical toilet comprising: a bowl for receiving excreta from a user; a seat for the user; a sensor for detecting a symptom of the user; having a user use the toilet to deposit excreta; detecting a symptom of the user with the sensor; transmitting data from the sensor to a processor; and using the data to measure the symptom and compare it to the user's normal or expected symptoms

Further aspects and embodiments are provided in the foregoing drawings, detailed description, and claims.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1 illustrates an analytical toilet with the lid closed, according to an embodiment of the disclosure.

FIG. 2 illustrates an analytical toilet with lid open, according to an embodiment of the disclosure.

FIG. 3 illustrates an analytical toilet with lid closed and a portion of the exterior shell removed, according to an embodiment of the disclosure.

FIG. 4 further illustrates the interior of the toilet of FIGS. 1-3, according to an embodiment of the disclosure.

FIG. 5 illustrates a modular analytical test device attached to a manifold, according to an embodiment of the disclosure.

FIG. 6 illustrates another embodiment of a modular analytical test device.

FIG. 7 illustrates another embodiment of a modular analytical test device.

FIG. 8 illustrates an analytical test device adapted for use with a microfluidic chip, according to an embodiment of the disclosure.

FIG. 9 illustrates an MFC analytic test device with an optical interface, according to an embodiment of the disclosure.

FIG. 10 illustrates an MFC analytic test device with a set of electrical contacts, according to an embodiment of the disclosure.

FIG. 11 illustrates an alternate configuration for a larger MFC analytic test device with additional standardized areas designated for fluidic, electrical, or optical interconnects, according to an embodiment of the disclosure.

FIG. 12 illustrates a detailed view of an exposure event of excreta to a sensor, according to an embodiment of the disclosure.

FIG. 13 is an isometric view of a toilet according to one embodiment according to the present disclosure.

FIG. 14 is a top view of the toilet of FIG. 1.

FIG. 15 is a view of the bottom of the seat and lid of the toilet of FIG. 1.

FIG. 16 is a view from the side of the toilet of FIG. 1 with the cover removed.

FIG. 17 is a view showing the top of a foot scale used in one embodiment of the invention.

FIG. 18 is a view of the bottom of the foot scale of FIG. 5.

FIG. 19 is an isometric view of a toilet used in another embodiment according to the present disclosure.

FIG. 20 is a top view of the toilet of FIG. 7.

FIG. 21 is a view of the bottom of the seat of the toilet of FIG. 7.

FIG. 22 is a partial view of the toilet of FIG. 7 with the cover removed.

FIG. 23 is a detail view of a handle used in an embodiment according to the present disclosure.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, “toilet” is meant to refer to any device or system for receiving human excreta, including urinals.

As used herein, the term “bowl” refers to the portion of a toilet that is designed to receive excreta.

As used herein, the term “base” or “frame” refers to the portion of the toilet below and around the bowl supporting it.

As used herein, the term “user” refers to any individual who interacts with the toilet and deposits excreta therein.

As used herein, the term “excreta” refers to any substance released from the body of a user including urine, feces, menstrual discharge, saliva, expectorate, and anything contained or excreted therewith.

As used herein, the term “excretion profile” is meant to refer collectively to the rate of excretion at any moment in time of an excretion event and the total volume or mass of excreta as a function of time during an excretion event. The terms “defecation profile” and “urination profile” refer more specifically to the separate measurement of excreta from the anus and urethra, respectively.

As used herein, the term “sensor” is meant to refer to any device for detecting and/or measuring a property of a person or of a substance regardless of how that property is detected or measured, including the absence of a target molecule or characteristic. Sensors may use a variety of technologies including, but not limited to, MOS (metal oxide semiconductor), CMOS (complementary metal oxide semiconductor), CCD (charge-coupled device), FET (field-effect transistors), nano-FET, MOSFET (metal oxide semiconductor field-effect transistors), spectrometers, volume measurement devices, weight sensors, temperature gauges, chromatographs, mass spectrometers, IR (infrared) detector, near IR detector, visible light detectors, and electrodes, microphones, load cells, pressure gauges, PPG (photoplethysmogram), thermometers (including IR and thermocouples), rheometers, durometers, pH detectors, scent detectors gas, and analyzers.

As used herein, the term “imaging sensor” is meant to refer to any device for detecting and/or measuring a property of a person or of a substance that relies on electromagnetic radiation of any wavelength (e.g., visible light, infrared light, x-ray) or sound waves (e.g., ultrasound) to view the surface or interior of a user or substance. The term “imaging sensor” does not require that an image or picture is created or stored even if the sensor is capable of creating an image.

As used herein, the term “data connection” and similar terms are meant to refer to any wired or wireless means of transmitting analog or digital data and a data connection may refer to a connection within a toilet system or with devices outside the toilet.

As used herein, “neural network”, “neural net”, and similar terms are meant to refer to a set of algorithms, modeled loosely after the human brain, that are designed to recognize patterns. They interpret sensory data through a kind of machine perception, labeling or clustering raw input. The patterns they recognize are numerical, contained in vectors, into which all real-world data, be it images, sound, text or time series, must be translated.

As used herein, the terms “biomarker” and “biological marker” are meant to refer to a measurable indicator of some biological state or condition, such as a normal biological processes, pathogenic processes, or pharmacologic responses to a therapeutic intervention. Some biomarkers are related to individual states or conditions. Other biomarkers are related to groups or classifications or states or conditions. For example, a biomarker may be symptomatic of a single disease or of a group of similar diseases that create the same biomarker.

As used herein, the term “analyte” is meant to refer to a substance whose chemical constituents are being identified and measured.

As used herein, the term “manifold” is intended to have a relatively broad meaning, referring to a device with multiple conduits and valves to controllably distribute fluids, namely water, liquid sample and air.

As used herein, the term “test chamber” is meant to refer broadly to any space adapted to receive a sample for testing, receive any other substances used in a test, and apparatus for conducting a test, including any flow channel for a fluid being tested or used for testing.

As used herein, a “fluidic circuit” is meant to refer to the purposeful control of the flow of a fluid. Often, this is accomplished through physical structures that direct the fluid flow. Sometimes, a fluidic circuit does not include moving parts.

As used herein, a “fluidic chip” is meant to refer to a physical device that houses a fluidic circuit. Often, a fluidic chip facilitates the fluid connection of the fluidic circuit to a body of fluid.

As used herein, the term “microfluidics” is meant to refer to the manipulation of fluids that are contained to small scale, typically sub-millimeter channels. The prefix “micro” used with this term and others in describing this invention is not intended to set a maximum or a minimum size for the channels or volumes.

As used herein, the term “microfluidics” is meant to refer to the manipulation of fluids that are contained to small scale, typically sub-millimeter channels. The “micro” used with this term and others in describing this invention is not intended to set a maximum or a minimum size for the channels or volumes.

As used herein, the term “microfluidic chip” is meant to refer to is a set of channels, typically less than 1 mm2, that are etched, machined, 3D printed, or molded into a microchip. The micro-channels are used to manipulate microfluidic flows into, within, and out of the microfluidic chip.

As used herein, the term “microfluidic chamber” is meant to refer to a test chamber adapted to receive microfluidic flows and/or a test chamber on a microfluidic chip. As used herein, the term “lab-on-chip” is meant to refer to a device that integrates one or more laboratory functions or tests on a single integrated circuit. Lab-on-a-chip devices are a subset of microelectromechanical systems (MEMS) and are sometimes called “micro total analysis systems” (μTAS).

As used herein, the prefix “nano” is meant to refer to something in size such that units are often converted to the nano-scale for ease before a value is provided. For example, the dimensions of a molecule may be given in nanometers rather than in meters.

As used herein, “miniaturized electronic system” is meant to refer to an electronic system that uses nanometer scale technology.

As used herein, “FET” is meant to refer to a field effect transistor, which is a device which uses an electric field to control the current flowing through a device. FETs are also known by the name “unipolar transistor”.

As used herein, “biomarker” and “biological marker” are meant to refer to a measurable indicator of some biological state or condition, such as a normal biological processes, pathogenic processes, or pharmacologic responses to a therapeutic intervention. Some biomarkers are related to individual states or conditions. Other biomarkers are related to groups or classifications or states or conditions. For example, a biomarker may be symptomatic of a single disease or of a group of similar diseases that create the same biomarker.

As used herein, “bind” and similar variants are meant to refer to the property of facilitating molecular interaction with a molecule, such as interaction with a molecular biomarker.

As used herein, “functionalize” and similar variants are meant to refer to a device, especially a nanometer scale device, the surface of which being configured to interact with a specific analyte, such as a specific biomarker.

As used herein, “genomic derived signal” is meant to refer to a molecule generated by the genome of a cell, bacteria, virus, or other nucleic acid carrier, such as DNA, RNA, microRNA, cell-free (circulating) nucleic acids or those of various immunologically related cells.

As used herein, an analyte that “interacts” with a sensor is meant to refer to several ways a component (e.g., receptor) of a sensor can detect the analyte. “Interacting” may include reversible binding of an analyte to a component in a sensor. This may also be referred to as labile binding where the analyte is weakly bound to a component in the sensor and can be removed by a removal treatment such as a flushing or cleaning process. “Interacting” may include irreversible binding of an analyte to a component in a sensor where the binding is a one-time event and the component of the sensor or the entire sensor must be replaced after each use. “Interacting” may include a non-binding event wherein the analyte is in the vicinity of the component of the sensor such that the magnetic, optical or electrical properties of the component are perturbed by the presence of an analyte. For example, this may be caused by negative or positive charges located on the surface of the analyte.

As used herein, “tremor”, “body tremor”, and similar variants are meant to refer to involuntary motion, particularly those related to repeated muscle contraction and relaxation leading to shaking movements in one or more parts of the body. This shaking may be considered rhythmic or cyclical because of the fairly consistent and repetitive motion of the tremor.

As used herein, “physiologic tremor” and its derivatives are meant to refer to a fine tremor resulting from normal body function such as heartbeat, maintaining a posture, and movement. Occurrence of these is normal and generally not cause for seeking additional health or wellness care.

As used herein, “exacerbated physiologic tremor”, “noticeable physiologic tremor”, and their variants are meant to refer to a physiologic tremor which has become more pronounced than normal and is generally an indication of factors which may warrant health or wellness consideration or care, such as extreme fatigue, stress, intense emotion, low blood sugar (hypoglycemia), an overactive thyroid, medications such as corticosteroids, amphetamines or beta-agonists, heavy metal toxicity, stimulants such as caffeine, fever, and alcohol withdrawal.

As used herein, “symptomatic tremor”, “abnormal tremor”, “atypical tremor”, and their variants are meant to refer to exacerbated physiologic tremors. They also include tremors whose existence is symptomatic, suggestive of, or correlates with an abnormal or atypical health or wellness condition, which condition may warrant additional care or consideration. It includes tremors that correlate with abnormal conditions and circumstances such as multiple sclerosis, stroke, traumatic brain injury, neurodegenerative diseases that affect parts of the brain (such as Parkinson's disease), use of certain medicines (including particular asthma medication, amphetamines, caffeine, corticosteroids, and some drugs used for psychiatric and neurological disorders), alcohol abuse or withdrawal, mercury poisoning, an overactive thyroid, liver or kidney failure, and anxiety or panic.

In general, “weight” refers to the force excreted by a physical object or organism, especially a person or animal, under the influence of a gravitational field. As used herein, “weight” is sometimes used to represent the more general term “force”, which represents the mass of the physical object or organism multiplied by the acceleration of that mass. On the surface of Earth, gravity applies a relatively constant acceleration to mass thereon, thus creating the force of weight people are generally familiar with. When measured, the weight or force itself is generally not directly measured, but reaction forces acting in opposition to the weight or force are being measured.

The force exerted by a tremor is created by motion of one part of a person's body relative to another part of the body. While gravity does not control amount of force, it may influence the final measurement of the force. Thus, the force exerted by a tremor may be measured in a gravity environment different from that of a person on the surface of the earth so long as the effects of environment's gravity are accounted for by the system. This includes environments termed “weightless” wherein the person or environment is in a state of falling relative to large gravity objects in the vicinity (such as being in a diving aircraft or in orbit around a planet), resulting in the sensation of being free of gravity. As such, the force being measured to detect a tremor from a person is more dependent on the person's movements than from the gravitational pull of Earth; this force may manifest as a temporary change to measured weight. Thus accelerometers, while not the simplest way to measure the weight of a person at rest on Earth, are an acceptable form of weight sensor to detect the cyclical loading and unloading of forces associated with tremors. In short, this disclosure is not meant to limit the invention to applications at rest on the surface of a planet or other environment with similar gravity.

As used herein, “foot support” and similar terms are meant to refer to a structure designed to receive force from a person's foot, feet, and/or lower leg(s). This includes a structure that limits a single degree of freedom, such as one that rests on a floor and feet are placed thereon, as well as a structure meant to limit multiple degrees of freedom, such as a foot, ankle, or lower leg restraint.

As used herein, “symptom” is meant to refer broadly to any physiological characteristic of a patient. The characteristic may be indicative of an injury, illness, or disease (e.g., presence of an infection, high or low body temperature, involuntary muscle tremors, high or low analyte levels) but is not limited to those. The characteristic may also be indicative of the user or non-use of a treatment including, but not limited to, medication, physical therapy, or exercise. Such characteristic may include analytes in the patient's system indicative of the user or non-use of medication. Symptom also includes side-effects of a medicament or other treatment.

### Exemplary Embodiments

The present disclosure relates to monitoring a patient's symptoms to detect changes. Changes in a patient's symptoms are used to monitor user compliance with treatment and the effectiveness of the treatment. There are many types of symptoms and ways to detect them. The present disclosure addresses a variety of symptoms and how they are measured and tracked.

The present disclosure relates to smart toilets with analytical tools (may also be referred to as an “analytical toilet” or a “health and wellness toilet”) which detect, analyze, and/or track the trends of analytes, such as biomarkers, of a user who deposits excreta into the toilet. More specifically, the toilet receives excreta from a user, processes the excreta in preparation for analysis, and brings a sample of excreta (including processed excreta) into a testing area for detection by nanometer scale circuitry component, BioFET, optical detector, or other similar testing components. The circuitry component has been functionalized to interact with a specific analyte, such as a biomarker, on a molecular or atomic level. The circuitry component provides a data signal depending on whether the specific analyte is present in the excreta sample in contact with it. After the toilet has finished with the excreta, the toilet purges the excreta from the toilet in preparation for receiving a new excreta sample.

In accordance with the present disclosure, an analytical toilet that includes an infrastructure for multiple health and wellness analysis tools is provided. This provides a platform for the development of new analytical tools by interested scientists and companies. Newly developed tests and diagnostic tools may be readily adapted for use in a system having a consistent tool interface.

In a preferred embodiment, the system will regularly query or receive information from other devices that obtain relevant information about the user. These devices may include smart scales, smart watches, smart phones (e.g., health and fitness apps), home pulse and blood pressure monitors, etc. The information may include health and wellness information, such as pulse, and location information (e.g., user at a gym). In a preferred embodiment, the system also obtains relevant information from healthcare providers via secure channels with permission of the user and provider.

In various exemplary embodiments, the analytical toilet provides a fluid processing manifold that collects and routes samples from the toilet bowl to various scientific test devices and waste handling portals throughout the device

In various exemplary embodiments, the analytical toilet provides multiple fluid sources via a manifold system. The manifold is adapted to connect to a plurality of analytic test devices adapted to receive fluids from the manifold. The manifold is designed to selectively provide a variety of different fluid flows to the analytical test device. These fluids may include, among others, excreta samples, buffer solutions, reagents, water, cleaners, biomarkers, dilution solutions, calibration solutions, and air. These fluids may be provided at different pressures and temperatures. The manifold and analytical test device are also adapted to include a fluid drain from the analytical test devices.

In various exemplary embodiments, the manifold system provides a standardized interface for analytical test devices to connect and receive all common supplies (e.g., excreta samples, flush water), data, and power. Common supplies may be supplied from within (e.g., reagents, cleaners) or without (e.g., water) the toilet system. The analytical test devices may be designed to receive some or all of the standardized flows. The analytical test devices may also include storage cells for their own unique supplies (e.g., test reagent).

In various exemplary embodiments, the manifold is adapted to direct fluids from one or more sources to one or more analytical test devices. The manifold and analytical test devices are designed such that analytical test devices can be attached to and detached from the manifold making them interchangeable based on the needs of the user. Different analytical test devices are designed to utilize different test methods and to test excreta samples for different constituents.

In various exemplary embodiments, the smart toilet provides an electrical power connection and a data connection for the analytical test device. In a preferred embodiment, the electrical power and data connections use the same circuit. In various exemplary embodiments, the toilet is provided with pneumatic and/or hydraulic power to accommodate the analytical test devices.

In various exemplary embodiments, the smart toilet platform performs various functions necessary to prepare samples for examination. These functions include, but are not limited to, liquidizing fecal samples, diluting or concentrating samples, large particle filtration, sample agitation, and adding reagents. Miniaturized mechanical emulsification chambers show promise for repeatable and sanitary stool preparation. Stool samples may also be liquefied using acoustic energy and/or pressurized water jets.

In various exemplary embodiments, the smart toilet also provides, among other things, fluid transport, fluid metering, fluid valving, fluid mixing, separation, amplification, storage and release, and incubation. The smart toilet also is equipped to provide cleansers, sanitizers, rinsing, and flushing of all parts of the system to prevent cross-contamination of samples. In some embodiments, the system produces electrolyzed water for cleaning.

In various exemplary embodiments, one layer of the fluidic manifold is dedicated to macro-scale mixing of fluids. Sample, diluents, and reagents are available as inputs to the mixers. The mixing chamber is placed in series with all other scientific test devices, allowing bulk mixed sample to be routed to anywhere from one to all stations (i.e., analytical test device interfaces) for analysis. Mixing may also occur in an analytical test device.

In various exemplary embodiments, samples are filtered for large particulates at the fluid ingress ports of the manifold. The fluid manifold uses a network of horizontal and vertical channels along with simple valves to route urine or prepared stool samples to one of several scientific test devices located on the platform.

In various exemplary embodiments, the manifold is constructed using additive layers, and different layers can be customized for particular applications. Standard ports and layouts are used for interfacing with external components, such as pressure sources and flow sensors. In general, characteristic channel volumes at the bottom of the manifold stack are on the order of milliliters. At the top of the manifold stack is the microfluidic science device, which will interface simultaneously with multiple microfluidic chips using standardized layout and pressure seals.

In various exemplary embodiments, the analytic test devices are designed to perform one or more of a variety of laboratory tests. Any test that could be performed in a medical or laboratory setting may be implemented in an analytical test device. These tests may include measuring pulse, blood pressure, blood oxygenation, electrocardiography, body temperature, body weight, excreta content, excreta weight, excreta volume, excreta temperature, excreta density, excreta flow rate, and other health and wellness indicators.

In various exemplary embodiments, the system is adapted to work with a variety of actuation technologies that may be used in the analytical test devices. The system provides electronic and fluidic interconnects for various actuator technologies and supports OEM equipment. In a preferred embodiment, the system is adapted to work with actuator modules that can be attached to the sample delivery manifold and controlled by a central processor. The system platform supports an inlet and outlet for the pressure transducer that interfaces with the fluidic manifold, and electronic or pneumatic connections where required. The system supports a variety of macro- and microfluidic actuation technologies including, but not limited to, pneumatic driven, mechanical pumps (e.g., peristaltic), on-chip check-valve actuators (e.g., piezo-driven or magnetic), electroosmotic driven flow, vacuum pumps, and capillary or gravity driven flow (i.e., with open channels and vents).

One benefit of the present disclosure is the detection, monitoring, and tracking of a user's biomarkers without having any inconvenience aside from what they would otherwise do using the toilet. Without the present disclosure, among other things, people often have to manually collect samples of excreta, use equipment they are less familiar with than a toilet, or wait longer for analysis and results. Each of these things can negatively impact a user's experience and/or the quality or accuracy of the results.

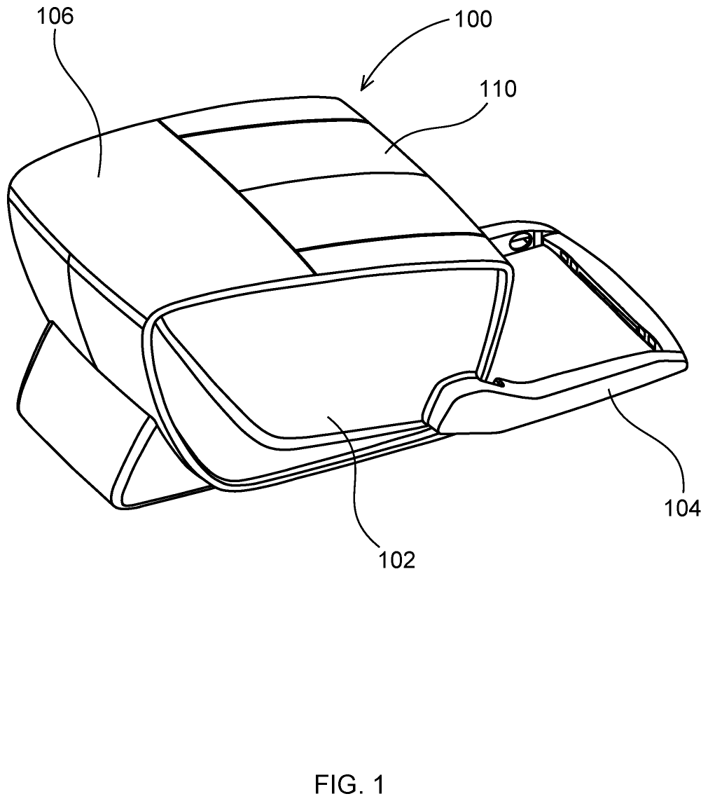

Now referring to FIGS. 1-3, a preferred embodiment of an analytical toilet 100 is shown. FIG. 1 illustrates the analytical toilet 100 with the lid 110 closed, according to an embodiment of the disclosure. FIG. 1 further shows exterior shell 102, foot platform 104 and rear cover 106. The lid 110 is closed to prevent a user from depositing excreta in toilet 100 until the toilet is ready for use.

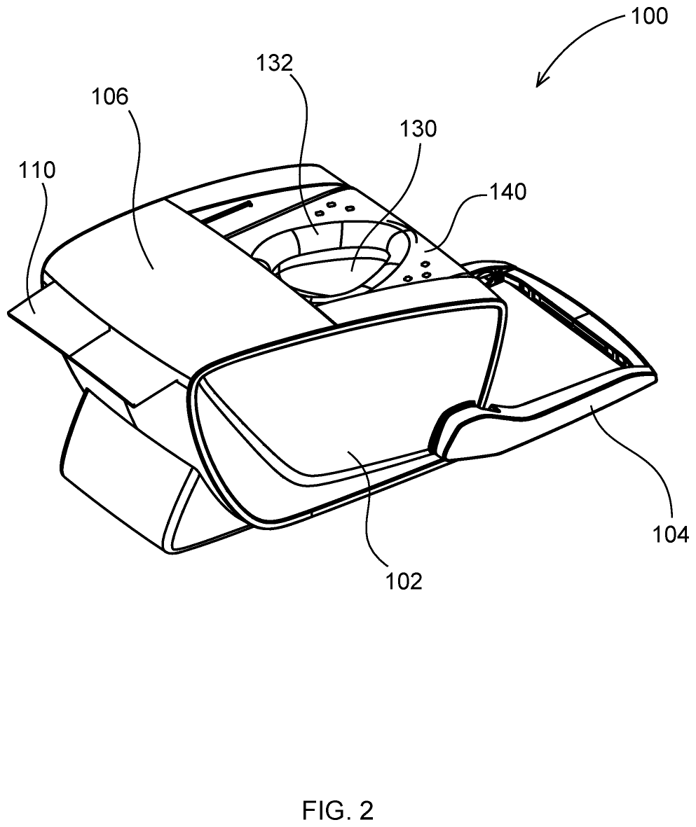

FIG. 2 illustrates toilet 100 with lid 110 open, according to an embodiment of the disclosure. Toilet 100 includes exterior shell 102, rear cover 106, bowl 130, seat 132, lid 110, fluid containers 140 and foot platform 104. Housed within toilet 100 are a variety of features, including equipment, that facilitate receiving excreta, processing excreta for analysis, analyzing excreta, and disposing of excreta. FIG. 2 shows toilet 100 with lid 110 open so a user can sit on seat 132 and deposit excreta in toilet 100.

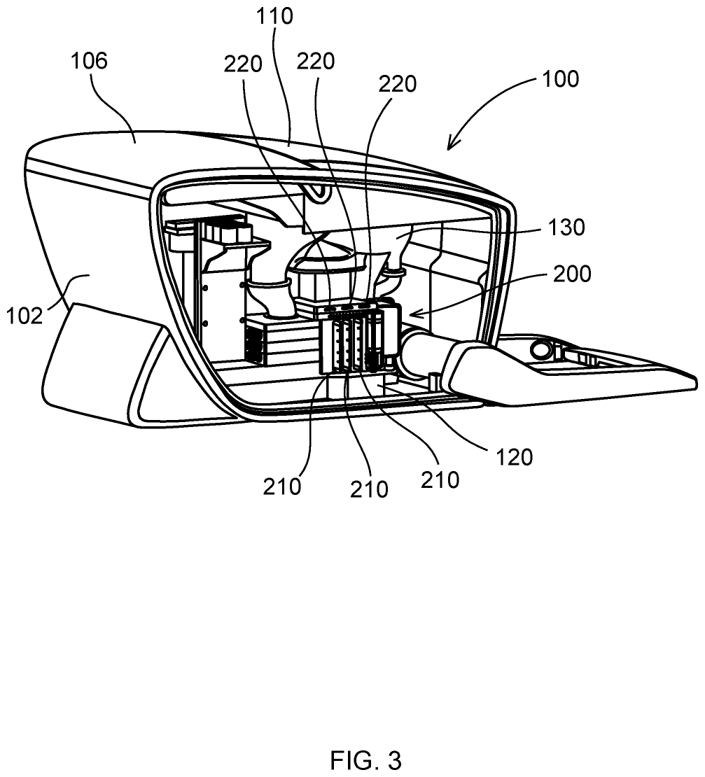

FIG. 3 illustrates toilet 100 with lid 110 closed and a portion of exterior shell 102 removed, according to an embodiment of the disclosure. This allows access to equipment housed within toilet 100. With exterior shell 102 removed, base 120 and manifold area 200 is visible. Manifold area 200 includes test areas 210 and fluidic chip slots 220. Preparation and/or analysis of sample can selectively take place in a test area 210 or fluidic chip slot 220. Manifold area 200 is the area where analysis takes place.

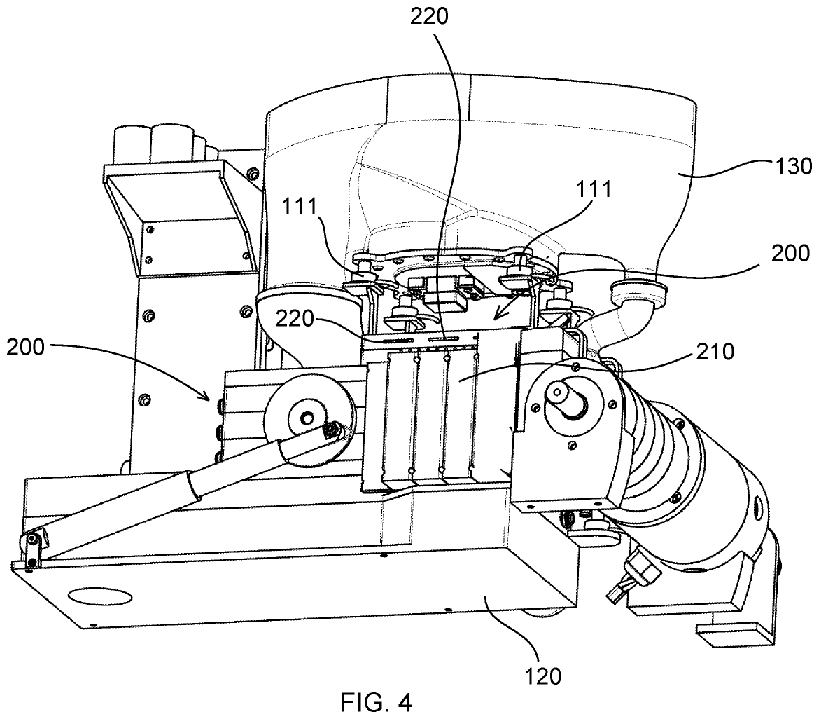

FIG. 4, further illustrates the interior of the toilet of FIGS. 1-3, according to an embodiment of the disclosure. The internal components of the toilet 100 are supported by a base 120. The bowl 130 is supported by one or more load cells 111. A manifold 200 is located below the bowl 130. The manifold 200 comprises a plurality of fluid paths. These fluid paths allow the manifold 200 to move fluids between the bowl 130, fluid containers 140, outside sources (e.g., municipal water supplies), other sources (e.g., air or water electrolyzing unit), analytical test devices 210, and the toilet outlet. The manifold 200 also provides electrical power and data connections to the analytical test devices 210. The manifold 200 can also directly pass fluids and/or solids from the bowl 130 to the toilet outlet.

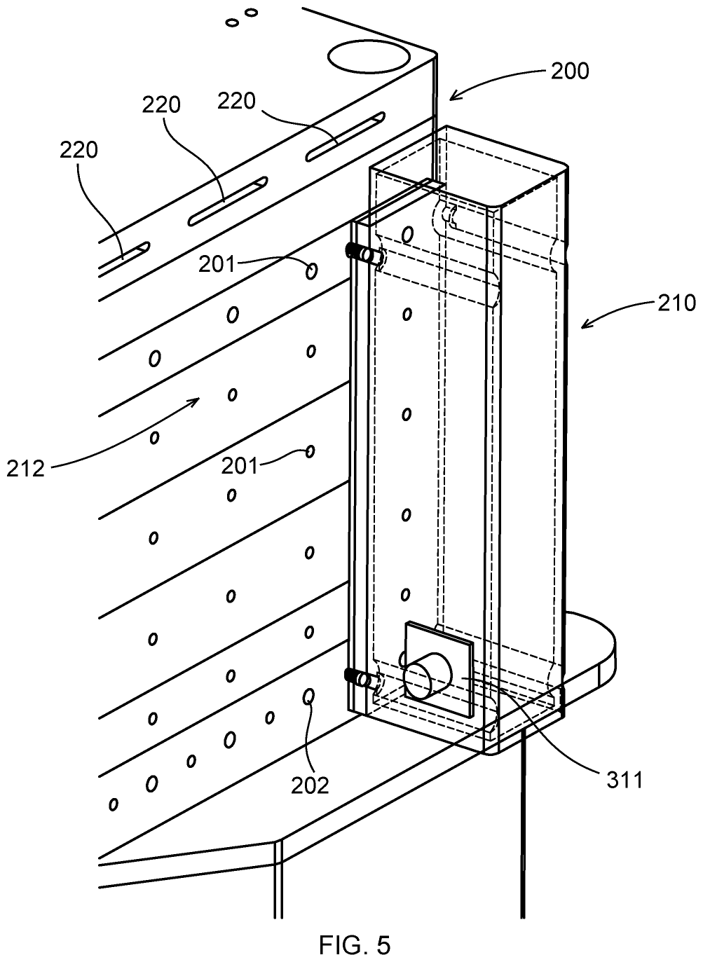

FIG. 5 illustrates a modular analytical test device 210 attached to a manifold 200, according to an embodiment of the disclosure. The manifold 200 is adapted to provide receptacles 210 with standardized connection interfaces for multiple analytical test devices 210. The manifold 200 is shown here with multiple fluid sources 201 for the analytical test device 210. In various embodiments, the manifold 200 may include receptacles 212 for more than one type of analytical test device 210 (e.g., different sizes, fluid supply needs, etc.). Slots 220 are also shown where microfluidic chips (MFCs) that further comprise sensor components may be inserted.

In various exemplary embodiment, the analytical test device 210 includes multiple inlets in fluid communication with the manifold 200. The selected fluid flows are directed into a test chamber with one or more sensors 311 (flow channels internal to the analytical test device not shown in FIG. 5). The sensors 311 may be one or more of electrochemical sensors, spectrometers, chromatography, CCD (charge-coupled device), or metal oxide semiconductor field-effect transistor (MOSFET) including complementary metal oxide semiconductor field-effect transistor (CMOSFET). The analytic test device 210 also includes at least one outlet 202 or drain in fluid communication with the manifold 200.

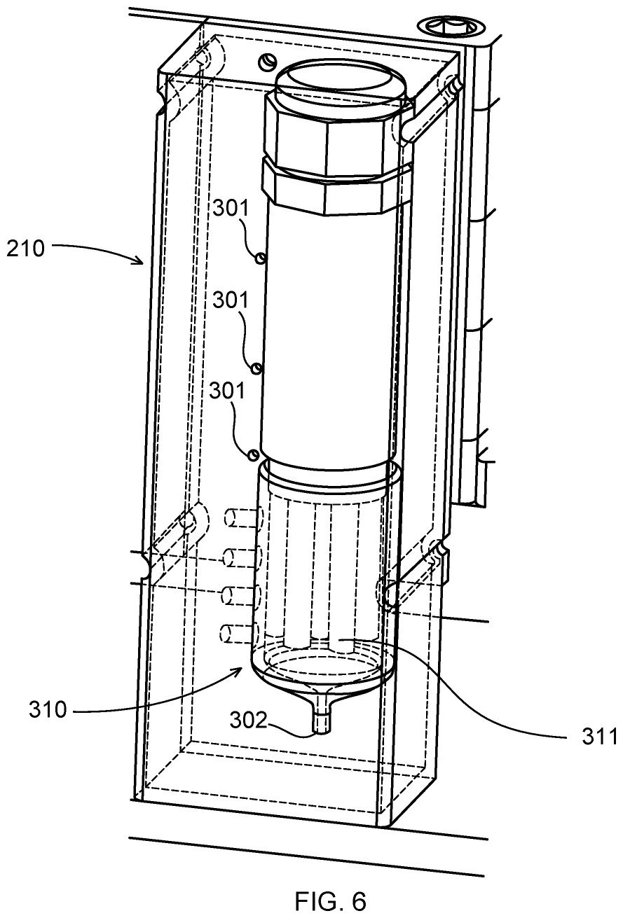

FIG. 6 illustrates another embodiment of a modular analytical test device 210. The analytical test device 210 includes multiple fluid inlets 301, test chamber 310, and at least one fluid outlet 302. The analytic test device 210 includes a test chamber 310 that received fluid flows and contains at least one array of sensors 311.

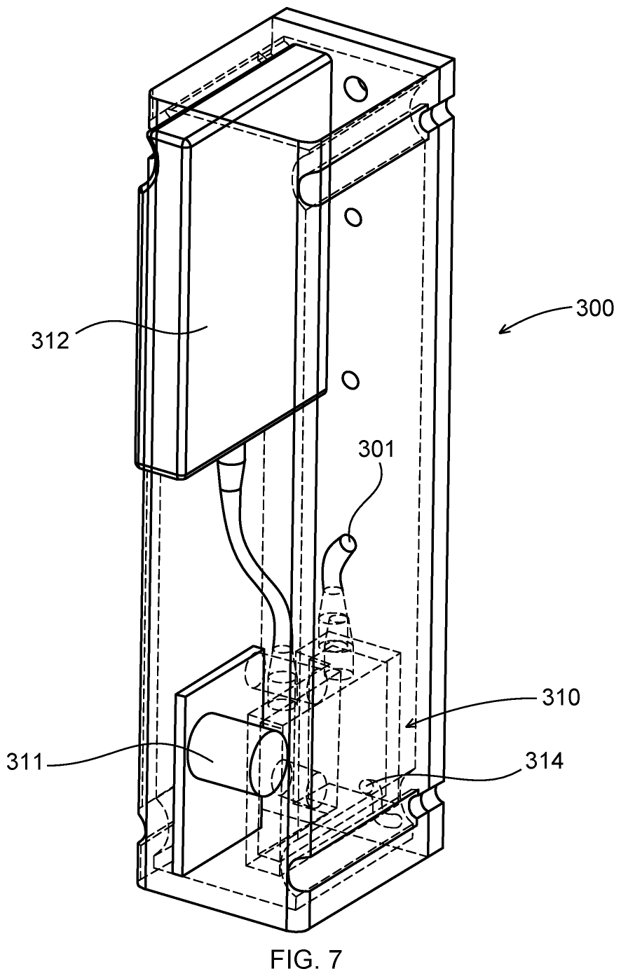

FIG. 7 illustrates another embodiment of a modular analytical test device 210. The analytical test device 210 includes multiple fluid inlets 301, test chamber 310, and at least one fluid outlet (not shown). This embodiment of an analytical test device 210 includes a storage cell 312, also in fluid communication with the test chamber 310. The analytical test device 210 may also include a pump to move fluid (e.g., test reagent) from the cell 312 to the test chamber 310. The analytic test device 210 also includes a camera adjacent to the test chamber 310 to monitor the contents of the test chamber 310. In various embodiments, the test chamber 310 is used to mix an excreta sample with a reagent that will cause a color change if a target analyte is present in the excreta sample. The camera is adapted to detect the color change. In various exemplary embodiments, the camera may be used to observe other characteristics or changes to the sample in the test chamber 310 (e.g., urine settling).

Once excreta has been deposited in the toilet, there are many ways it could be processed in preparation for testing and disposal. Some pretreatments include a filter, a centrifuge, dilution, or pH normalization. In one preferred embodiment, a portion of feces is separated from urine, mixed with water and/or a reagent, and presented to the component of a sensor for analysis. Following analysis, the sample is removed from the sensor, and the sensor is cleaned and/or sterilized in preparation for a new sample being presented to the component of the sensor.

There are many ways to incorporate the sensor into the toilet, the selection of which will depend on various factors, including ease of manufacture and maintenance, target market, physical constraints, frequency of use compared to other desired functions of the toilet, and cost. In one preferred embodiment, the sensor is built into a fluidic circuit. More preferably, the fluidic circuit is on a fluidic card. Still more preferably, the fluidic circuit on the fluidic card is a microfluidic circuit on a micro fluidic card. Preferably, the fluidic card is inserted into a slot or receptacle of the toilet which connects the fluid circuit on the card to the toilet's fluidic delivery system, enabling the card to receive the sample derived from the excreta. Alternatively, the sensor is part of a larger device that may be attached to the toilet, such as a device that processes and/or analyzes excreta. Alternatively, the sensor is built into the toilet rather than being on a card. Alternatively, the sensor is external to the remainder of the toilet and is connected to receive and/or return fluid from the toilet, such as may be accomplished by connecting the sensor to part of the toilet with tubes or pipes.

In a preferred embodiment, the sensor is functionalized to interact with a biomarker and produce a signal based on the presence and/or concentration of the biomarker. Often, this means the sensor is configured to respond to an individual molecule or even a specific molecular element or portion of a biomarker. Biomarkers that work well with this kind of sensor include immunological genomic derived signals, DNA genomic derived signals, RNA genomic derived signals, microRNA genomic derived signals, other genomic derived signals, proteins, carbohydrates, lipids, metabolites, and ionic concentrations. In some preferred embodiments, the component of the sensor amplifies the concentration of the targeted analyte. In other preferred embodiments, the component dilutes the concentration of the targeted analyte. In some preferred embodiments, the concentration is neither amplified nor diluted. Use of one category of tests to detect a particular analyte does not preclude use of another test category to detect or measure the same analyte.

It can be useful to group biomarkers. There are a variety of relevant groupings. For example, some groupings worth being considered are as follows: ionic or electrochemical, immunological, chromogenic, labeling or biotinylation, fluorescent binding, staining reactions, and transfection or genotypic. Regarding these groupings, the following list is not exhaustive but instead are select examples:
Ionic and electrochemical:
   The pH level can be useful as abnormal values are indicative of medical issues. It can be used to normalize urine concentration.
   Amperometry may be measured via ion selective membranes. Specific biomarkers to look for include calcium, sodium, and potassium.
   Detection of alpha-methylacyl-CoA racemase is helpful in the detection of prostate cancer.
Immunological
   Finding Interleukin-8 level helps with the detection of protein overabundance, which can indicate infection.
   Detection of Albumin in urine is indicative of kidney disease.
   Detection of creatinine, a protein metabolite, is indicative of whether the liver is functioning properly. It can be used to calibrate other test results, e.g. Albumin.
   Thrombin is indicative of blood clotting in the kidneys.
   Prostate cancer can be detected by testing for a prostate specific antigen.
   Detecting HIV p24 antigen helps in the detection of HIV disease
   Neutrophil gelatinase-associated lipocalin (NGAL), a marker for renal disease, helps track disease progression and effectiveness of treatment.
   With a color reagent, the resulting colorimetric data can be indicative of a variety of conditions.
   Biliruben is indicative of gall stones, infection, and/or liver malfunction.
   Urobilinogen is indicative of liver diseases.
   Nitrites are indicative of a urinary tract infection (UTI)
   Ketones are useful in monitoring metabolism.
Chromogenic
   E. coli detection helps determine if there's a bacterial infection.
   Thrombin detection is helpful as its presence in urine may indicate existence of a blood clot.
Labeling, biotinylation
   Glucose detection and levels are helpful in understanding a variety of conditions and states.
   Detection of C-polysaccharide can be useful in determining pneumonia, respiratory infections, treatment effectiveness.
Fluorescent binding
   Cancer can be detected using a general tool for fluorescein functionalized binding agents.
   Albumin (as noted above)
   Urinary metabolites are indicative of a variety of states and conditions.
   Coronavirus (severe acute respiratory syndrome (SARS), middle eastern respiratory syndrome (MERS), coronavirus disease 2029 (Covid-19)) cDNA fragments can be detected through DNA binding.
   TNF-alpha detection is indicative of autoimmune disorders.
Staining reactions
   Urine cytology can be helpful. It is achieved through a variety of methods, including automated microscopy or Al driven cytology.
Transfection, genotypic
   DNA from tumors, gene targeting, and RNA expression can be determined through various assays.

There are many variations on sensors that detect biomarker molecules or atoms that may be used in a health and wellness analytical toilet described herein.

### FET-Based Sensors

One exemplary class of sensors are biosensor field-effect transistors (BioFETs). BioFETs are based on metal-oxide-semiconductor field effect transistors (MOSFETs) that are gated by changes in the surface potential induced by the binding of biomolecules. Complimentary metal-oxide-semiconductor field effect transistors (CMOSFETs) may also be used. BioFETs comprise a field effect transistor and a biological recognition element or receptor.

BioFET-based sensors for a health and wellness analytical toilet may comprise one or more nanowires or functionalized nanowires to bind with a biomarker, one or more nanocrystals or functionalized nanocrystals, one or more sheets of graphene or functionalized graphene or a combination thereof. These materials are placed in a manner in the FET to bridge the source and drain electrodes. The BioFET may comprise a semiconductor with a functionalized gate. Other sensors include colorimetric based assays, paper-based analytical devices, a luminescent markers or labels, and a fluorescent or otherwise optically stimulated marker or label.

In some embodiments, nanowires for use in BioFETs may include conducting polymers such as polythiophene, polyaniline, polycarbazole, poly(3,4-ethylenedioxythiophene), polypyrrole, polyphenol or combinations thereof. Nanowires may comprise metals such as germanium, silver, gold, platinum, nickel palladium or combinations thereof. Nanowires may comprise two or more metals in a core-shell like arrangement. The metallic nanowires may comprise a thin oxide surface layer for covalent attachment of biomarker receptors. Nanowires may include inorganic oxide materials such as indium oxide (In₂O₃), indium tin oxide (ITO), zinc oxide (ZnO), tin oxide (SnO), titania (TiO₂) or silica (SiO₂). In an exemplary embodiment, the nanowire comprises a non-functionalized or functionalized single walled carbon nanotube (SWCNT) or a non-functionalized or functionalized multi-walled carbon nanotube (MWCNT) or a combination thereof. In a more exemplary embodiment, the nanowire comprises silicon (Si). The Si nanowire may comprise p-type or n-type Si. The Si nanowires may have a diameter of about 2 nm or larger. In other embodiments, the Si nanowires may have a diameter of about 2-100 nm. In an exemplary embodiment, the diameter of the Si nanowire may be in the range of about 2-30 nm. The nanowire used may have an aspect ratio of length to diameter in a range of about 500-1500. The nanocrystals may comprise colloidal metal, such as gold, or quantum dots. The nanocrystals may comprise semiconducting or super paramagnetic metal oxides such as iron oxides. Some variations include multiple sensors per component that detect the same biomarker, diverse concentration strengths of the same biomarker, and combinations of multiple biomarkers in an array or assay panel.

The conducting polymers, nanowires and nanocrystals used in FET-based sensors for use in a health and wellness analytical toilet described herein may be exploited for their optical, magnetic and electrical properties to detect various analytes. Their optical, magnetic and electrical properties may be tuned based on their size, how they are made, composition and how they are functionalized. A variety of transduction methods may be used to convert a binding event of a biomarker to a component in a sensor to a detectable and monitorable digital signal. The digital signal may comprise conductivity, resistance, voltage, conductance, fluorescence, spectroscopic, pH, magnetic changes or a combination thereof. In an exemplary embodiment, conductance or voltage or both the conductance and voltage in a FET-based sensor may be monitored when sensing for a biomarker. The conductance or voltage or both the conductance and voltage may be monitored with respect to time when a biomarker interacts with the sensor.

In some embodiments, conducting polymers, nanowires and nanocrystals used in FET-based sensors for use in a health and wellness analytical toilet described herein may be functionalized with one or more monoclonal antibody receptors. The receptors may be covalently attached. Antibody receptors may be used to detect one or more viruses. Such viruses may include DNA, RNA or reverse transcribing viruses. An individual sensor may comprise only one type of antibody to target and detect a specific virus, such as influenza A, adenovirus, covid-19 or ebola. In other embodiments, a sensor may comprise two or more antibodies to target and detect two or more different types of viruses.

In other embodiments, conducting polymers, nanowires and nanocrystals used in FET-based sensors for use in a health and wellness analytical toilet described herein may be functionalized with one or more monoclonal antibodies to detect pathogens that cause diseases such as cancer. Cancerous tumor cells release antigens that can be detected. These antigens may be proteins, peptides or polysaccharides. In an exemplary embodiment, a FET-based sensor in an analytical toilet may comprise one or more antibodies to detect antigens released by cancerous cells. Antigens are biomarkers released by cancerous cells may also be referred to as tumor markers. Such biomarkers may include CA 15-3 from breast cancer cells. Prostate specific antigen (PSA) found in prostate cancer cells. CA-125 antigen biomarker commonly found in ovarian cancer cells. Carcinoembryonic antigen (CEA) found in colorectal cancer cells.

Other biomarkers that may be detected by a FET-based sensor in an analytical toilet described herein include leucine-rich α-2-glycoprotein (LRG1), isoform-1 of multimerin-1 (MMRN1), S100 calcium-binding protein A8 (S100A8), serpin B3 (SERPINB3) and differentiation-44 antigen (CD44) for cervical cancer. Biomarkers bladder-tumor-associated antigen, nuclear matrix protein 22 (NMP22), Calreticulin, clusterin, cystatin B, proepithelin, UHRF1, bladder tumor antigen (BTA), human complement factor H related protein (hCFHrp), nuclear matrix protein 22 (NMP22), angiopoeitin (ANG), apolipoprotein E (APOE), interleukin-2 (IL-2), interleukin-6 (IL-6), interleukin-8 (IL-8), interleukin-10 (IL-10), interleukin-12 (IL-12), interleukin-18 (IL-18), interleukin-1ra (IL-1ra), TNF-α, MMP-9, MMP-10, plasminogen activator inhibitor 1 (PAI-1), Semenogelin-2, Vascular endothelial growth factor (VEGF), Coronin-1A, DJ-1, PARK7, Gamma Synuclein, Apo-A1, UP1, soluble Fas, ORM1, HtrA1, hyaluronidase, CP1, CCL18, BLCA-4and α-1B-glycoprotein for bladder cancer. Collagen α-1(III) peptide, collagen α-1(I) peptide, TMPRSS2-ERG and PCA3 biomarkers for prostate cancer. Cathepsin D, NMP22, creatinine, microalbumin, sodium (Na) and potassium (K) biomarkers for renal cancer. Eosinophil-derived Neurotoxin C-terminal Osteopontin fragments and Bcl-2 biomarkers for ovarian cancer. Matrix metallopeptidase-9 (MMP-9), HER-2 and ADAM12 biomarkers for breast cancer. Cystatin SN biomarker for colorectal cancer. IL-6, MMP9 biomarkers for multiple myeloma. It should be noted that some biomarkers are indicative of more than one type of cancer. It also should be noted that this is not an exhaustive list.

Some biomarkers may be indicative of injury or trauma. For example, a FET-based sensor in an analytical toilet described herein may be used to detect at least one biomarker released as a result of a kidney injury. These include urinary neutrophil gelatinase-associated lipocalin (NGAL), cystatin C (CyC), clusterin (CLU), hepatocyte growth factor (HGF), π-glutathione-S-transferase (π-GST), α-GST, kidney injury molecule-1 (KIM-1), osteopontin (OPN), renal papillary antigen (RPA-1), albumin, β2-microglobulin, trefoil factor-3 or urea.

A FET-based sensor in an analytical toilet described herein may be used to detect at least one biomarker released as a result of cardiovascular disease. This includes N-terminal pro-BNP (NT-proBNP), C-type natriuretic peptide (CNP), mRNA in urine supernatant (US-mRNA), adrenodoxin (ADX), eosinophil cationic protein (ECP), fetuin B (FETUB), growth differentiation factor 15 (GDF15), guanine deaminase (GUAD) or neurogenic notch homolog protein 1 (NOTCH1).

A FET-based sensor in an analytical toilet described herein may be used to detect at least one biomarker released as a result of a brain disease. This includes azelaic acid, N-methylnicotinamide, α-hydroxybutyrate, choline, formate, and N-methylnicotinamide, oxaloacetate or acetone biomarkers for bipolar disorder. Taurine, glutamate, N-acetyl glycoprotein, 3-(3-hydroxyphenyl)-3-hydroxypropanoic acid, five-carbon sugars, ribose, fructose, 1,2,3-butanetriol and propylene glycol biomarkers for autism. Glucosamine, glutamic acid, vanilmandelic acid, creatinine, α-ketoglutaric acid (α-KG), citrate, valine and glycine for schizophrenia. Serum albumin, apolipoprotein A-I, heparan sulfate proteoglycans (HSPGs), malonate, N-methylnicotinamide, m-hydroxyphenylacetate, hippuric acid, quinolinic acid or tyrosine biomarkers for depression.

A FET-based sensor in an analytical toilet described herein may also be used to detect at least one biomarker released as a result of various diseases such as fibrinogen for chronic obstructive pulmonary disease (COPD) or galactomannan for invasive aspergillosis.

A FET-based sensor in an analytical toilet described herein may also be used to detect nitrites for the presence of bacterial cystitis. Bacterial cystitis is commonly referred to as urinary tract infection (UTI). A FET-based sensor in an analytical toilet described herein may also be used to detect ketones such as acetoacetate (AcAc), Acetate (Acetone) or Beta-hydroxybutyrate (BHB) for the presence of diabetic ketoacidosis (DKA).

A component in a FET-based sensor in an analytical toilet described herein may be functionalized with peptide nucleic acid (PNA). PNA can be used as a gene sensor. A PNA is a non-charged variant of DNA and has high selectivity toward complementary DNA sequences. A PNA sensor is very sensitive with almost no electrochemical response toward DNA with one base mismatch. A PNA-based sensor may be used for detection of the DNA sequence responsible for sickle cell anemia.

A FET-based sensor in an analytical toilet described herein may be able to detect one or more viruses. The detectable viruses may be from the coronavirus class including alphacoronoavirus, betacoronovirus, gammacoronavirus or deltacoronavirus. More specifically, these viruses may include SARS-CoV-2 (also known as COVID-19) or SARS-CoV. A component in a FET-based sensor may be functionalized with angiotensin converting enzyme 2 (ACE2) antibody as a receptor. The ACE2 receptor interacts with the spike protein on the surface of the SARS-COV-2 virus.

In an exemplary embodiment, sensors used in a health and wellness analytical toilet described herein are capable of detecting non-biomarker molecules. Such molecules may comprise prescription drugs, recreational drugs, or illicit drugs. These may include amphetamines, nicotine, cannabinoids, opioids, cocaine, heroin, ethanol, methanol, pharmaceuticals, or other various stimulants or depressants.

In an exemplary embodiment, sensors used in a health and wellness analytical toilet described herein are capable of multiplexed detection. Multiplexed detection is necessary for simultaneous detection of multiple biomarkers such as proteins. This is critical for reliable detection of complex diseases such as cancer. In some embodiments, FETS comprising both n-type and p-type Si nanowires with different receptors within the same sensor may be required for reliable cancer and other disease detection.

In some instances, the high ionic strength environment in a health and wellness analytical toilet from excreta may adversely affect the accuracy and precision of the FET-based biomarker sensor. In some embodiments, a biomolecule permeable layer may be located over the sensor. The biomarker permeable layer may be substantially impermeable to ions such that only biomarkers are able to pass through the layer and approach the sensor. The layer may increase the effective Debye screening length in the region immediately adjacent to the sensor surface. This may allow detection of biomolecules in high ionic strength solutions in real-time. In some embodiments, the layer may only be permeable to a target analyte. In some embodiments, the layer may be only permeable to a class of analytes. The layer may be comprised of a membrane. The layer may be porous. The layer may be comprised of a polymer. The polymer may be comprised of polyethylene glycol.

In some embodiments, BioFET-based sensors in a health and wellness analytical toilet may be combined with other methods of biomarker detection. Additional biomarkers may be measured via a miniaturized mass spectrometer. Alternatively, additional biomarkers may be measured using gas chromatography integrated into the toilet body or positioned adjacent to the toilet. Additional biomarkers may also be measured using fluorescence spectrometry. A fluorescent tag may be covalently or ionically attached to a target molecule. These tags may be a protein, antibody, peptide or amino acid. These tagged molecules may then be used to detect a specific target such as an antigen. In some instances, two or more detection methods, such as those described herein, may be used to detect the same biomarker.

In various exemplary embodiments, microfluidic systems may be used to isolate and transport a sample, add and mix reagents if appropriate, and test the sample for one or more biomarkers on a small scale (i.e., sub-millimeter scale) in a health and wellness analytical toilet described herein. The microfluidic system may comprise an open microfluidic system, continuous-flow microfluidic system, droplet-based microfluidic system, digital microfluidic system, nanofluidic system, paper-based microfluidic system or combinations thereof.

A microfluidic-based biomarker detection system may be located on a microfluidic chip (MFC). In a preferred embodiment, the MFC includes a test chamber with a lab-on-chip (“LoC”) (also known as “test-on-chip”). The LoC may be designed to perform one or more laboratory tests. In various exemplary embodiments, one or more microfluidic chips (MFCs) may be removed or added to the toilet system as desired or needed at any given time, such as for different biomarker tests. In an exemplary embodiment, a DNA microfluidic chip may be used as a component in a biomarker sensor in a health and wellness analytical toilet. The DNA chip may comprise a DNA microarray, such as the GenChip DNAarray (Affymetrix, Santa Clara, Calif., USA). The DNA microarray comprises one or more pieces of DNA (probes) for biomarker detection. The MFC may comprise one or more affixed proteins in an array-like fashion. In an exemplary embodiment, the proteins are monoclonal antibodies for detection of antigens.

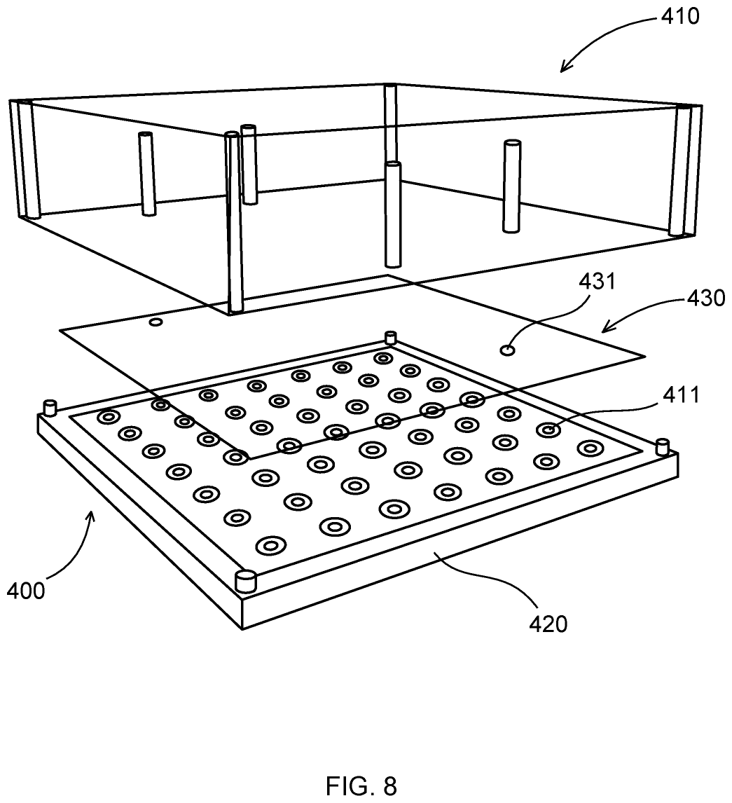

FIG. 8 illustrates an analytical test device 400 adapted for use with a microfluidic chip (MFC), according to an embodiment of the disclosure. The MFC analytical test device 400 includes a test chamber 410 with a lab on chip 411. The LoC 411 may comprise one or more sensor components previously described herein, such as one or more BioFETs. The manifold 200 includes a plurality of slots 220 or other openings for placement of a MFC analytical test device 400. An interface 420 with multiple ports 421, which act as fluid inlets or outlets for the test chamber 410, to provide connections and/or supplies for the MFC analytic test device 400. Protrusions 422 encircle the ports 421 to provide a point of positive contact for the sealing gasket 430, minimizing dead volume. Pores 431 in the gasket 430 select or block possible interactions with the MFC analytical test device 400.

In various exemplary embodiments, the backplate interface 420 is machined or molded with multiple microfluidic pores 421 for ingress of fluids or for removal of fluids. The interface 420 can be used with a variety of MFC analytic test device 400 testing modules. The ports 421 are preferably sealed by placing a gasket 430 between the MFC analytic test device 400 and backplate 420. The gasket 430 may be designed with pores 431 to selectively allow fluid flow through selected ports 421 and block potential flow through others depending on the MFC analytic test device 400 design. The gasket 430 may have alignment holes or features, with matching structures in the interface 420 or MFC analytic test device 400, to facilitate aligning the sheet of material to the pores 431. For example, the gasket 430 may fit snugly in a recess in the interface 420, or alignment pins in the interface 420 may match holes patterned in the gasket 430 by the same process used to create the pores 431. The gasket 430 may have corrugations, or variations in thickness, or be composed of multiple layers of different materials designed to reduce distortions propagating from one point of contact to another, in order to improve the alignment of the gasket 430 to the pores 431 during installation or when the system is pressurized.

In various exemplary embodiments, the gasket 430 may function as a seal for otherwise open channels in the MFC analytic test device 400, permitting pressurized flow in those channels. The gasket 430, typically an electrical insulator, may have electrical contacts built into the top, bottom, or intermediate layers of material, or embedded within the gasket 430 material. The gasket 430 may have electrical contacts built to cause a voltage potential to exist in the fluid. The gasket 430 may have electrical contacts built on the surface to come into contact with the fluid or gas and provide a potential to create electrochemical interactions. The gasket 430 may have electrical contacts built on the surface to come into contact with the fluid or gas and measure the electrical potential or ionic current.

In various exemplary embodiments, when the MFC analytic test device 400 is mechanically clamped to the plate 420 with sufficient pressure, the gasket 230 material creates a high-pressure seal between the MFC analytic test device 400 and the interface 420. Pores 431 in the gasket 430 open a channel between the backplate 420 and the MFC analytic test device 400. The gasket 430 material may be optimized for the application, including selecting chemically inert material. Alternatively, each pore 431 may be circumscribed with an elastomer O-ring that provides a seal under pressure.

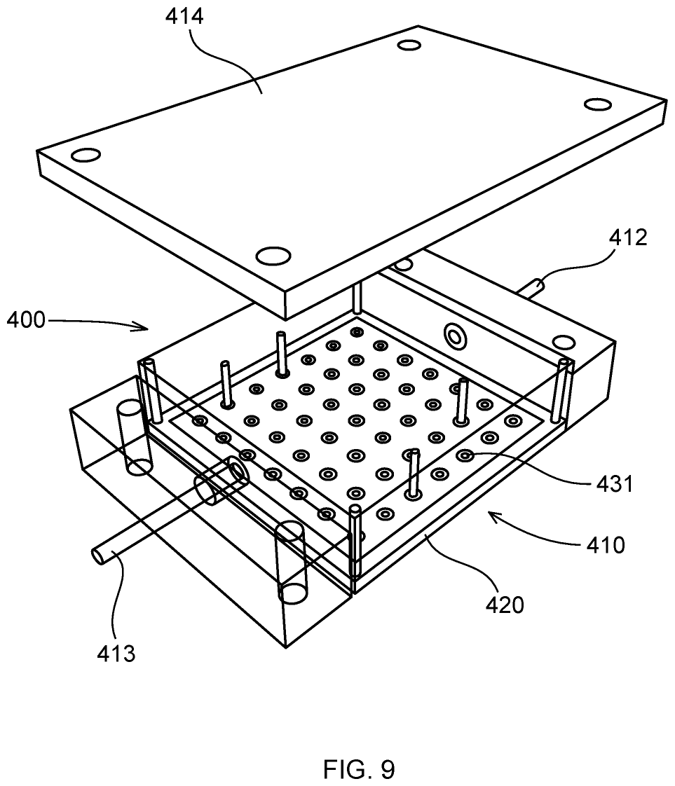

FIG. 9 illustrates an MFC analytic test device 400 with an optical interface, according to an embodiment of the disclosure. In some embodiments, a light source and light detector are connected to the test chamber 410 by fiber optic cables 412 and 413 are part of the test chamber 410 (e.g., spectrometer). The MFC analytical test device 400 is attached to the interface and held in place by a clamp plate. In an alternative embodiment, the light source may be placed in the clamp plate 414 to deliver light to a standardized location normal to the MFC analytic test device 400.

In various exemplary embodiments, the light detector may include various mechanisms for reducing reflections, such as covering the detector with an anti-reflection coating, film. The light detector may be an optical waveguide or other photonic sink.

In various exemplary embodiments, the MFC analytical test device 400 is secured to the interface 420 by a clamp 414. In a preferred embodiment, the clamp 414 serves a dual purpose as a back-side interface for microfluidic ports 421 in the reverse side of the MFC analytic test device 400. Microchannels machined into the clamp 414 in standardized locations may be included in the clamp design.

In various exemplary embodiments, the microfluidic fixtures, including screw-in connectors, may interface with micro-tubing to provide a connection elsewhere in the system, or back to the same chip. The micro-tubing may provide chip-to-chip connections.

In various exemplary embodiments, the clamp may serve as a housing or mounting point for a mirror that directs laser light through the MFC analytic test device 400. Some implementations may use a semi-transparent mirror that allows several MFC analytic test devices 400 arranged in a line to use the same laser beam to interact with MFC analytic test device 400 components. The platform microfluidic interface/manifold provides ports intended for this purpose.

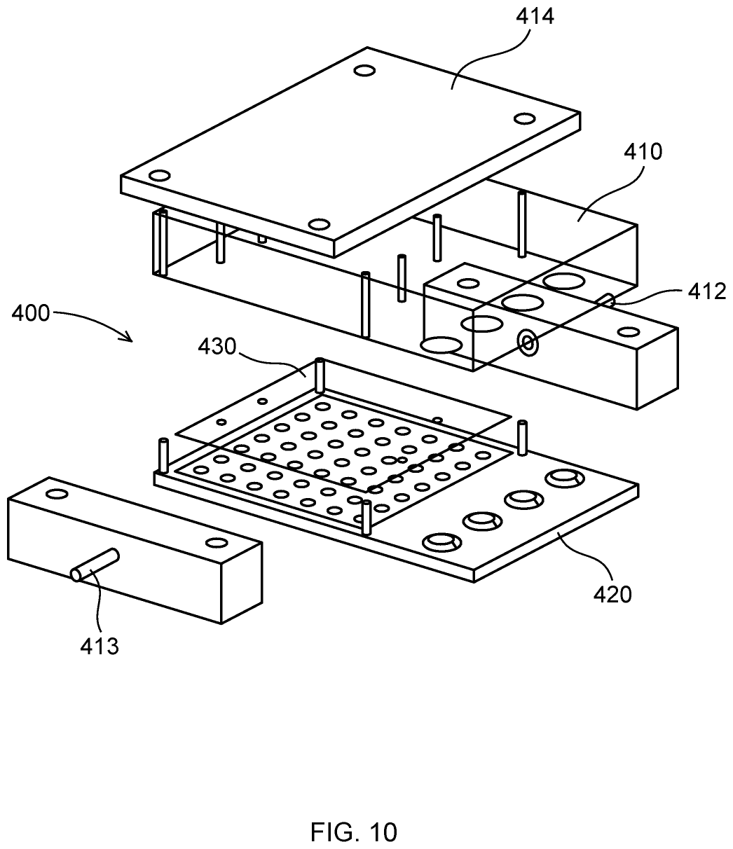

FIG. 10 illustrates an MFC analytic test device 400 with a set of electrical contacts, according to an embodiment of the disclosure. Electrical pads in a standardized location are made available for generic electrical operations, such as providing high and low voltage contacts, control signals, and ground pins. Corresponding pads are also located on the MFC analytic test device 400.

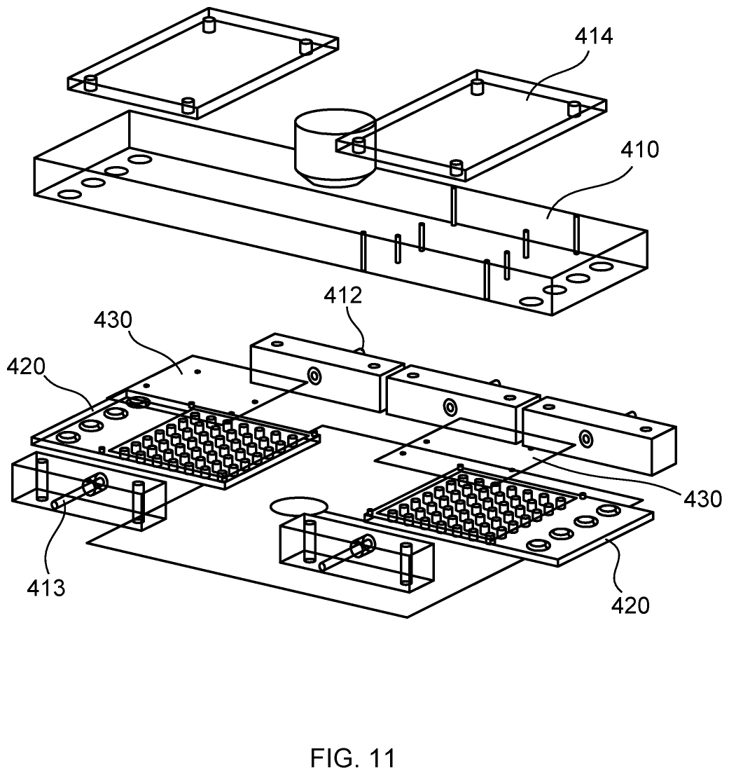

FIG. 11 illustrates an alternate configuration for a larger MFC analytic test device 400 with additional standardized areas designated for fluidic, electrical, or optical interconnects, according to an embodiment of the disclosure. This embodiment shows a designated viewing area, where a light source illuminates the MFC analytic test device 400 from below, and an image detector (possibly including a microscope or other magnifier) examines the output. Other embodiments may include an electronically controlled shutter that selectively blocks the light or selects a pinhole/orifice size for the light. The light source may be visible, UV, or other wavelength range of light emissions. The light source may be wide-band or narrow band.

In various exemplary embodiments, the MFC analytical test device 400 is designed to use very small quantities of reagent. In various exemplary embodiments, reagents are dispensed using technology similar to that used in inkjet printers to dispense ink. In some embodiments, an electrical current is applied piezoelectric crystal causing its shape or size to change forcing a droplet of reagent to be ejected through a nozzle. In some embodiments, an electrical current is applied to a heating element (i.e., resistor) causing reagent to be heated into a tiny gas bubble increasing pressure in the reagent vessel forcing a droplet of reagent to be ejected.

In various exemplary embodiments, the toilet fluidic manifold provides routing. Interconnecting levels of channels allows routing from one port to all others. Each channel includes an accumulator; allows for constant pressure pumping of all active channels simultaneously, while time-multiplexing pump-driven inflow.

In various exemplary embodiments, the manifold has reaction chambers built in for general purpose mixing operations. Each chamber has a macro-sized channel through which the manifold delivers a urine sample (filling the reaction chamber), and the chamber has a micro-sized channel. Pumps located internal or external to the manifold drive fluid into the reaction chamber, and into the micro-sized channel. A valve at the output of the macro-channel, and possibly at the output of the micro-channel, controls fluid direction as it exits the reaction chamber.

Microfluidic applications require support infrastructure for sample preparation, sample delivery, consumable storage, consumable delivery or replenishment, and waste extraction. In various exemplary embodiments, the manifold includes integrated support for differential pressure applications, pneumatic operations, sample and additive reservoirs, sample accumulators, external pumps, pneumatic pressure sources, active pump pressure (e.g., peristaltic, check-valve actuators, electro-osmotic, electrophoretic), acoustic or vibrational energy, and light-interaction (e.g., spectrometer, laser, UV, magnification).

In various exemplary embodiments, the manifold interface has a matrix of ports, possibly laid out in a regular grid. These ports may be activated or closed via an external support manifold. Routing is fully programmable.

In various exemplary embodiments, the manifold directs one or more fluids to the analytical test device 210 or MFC analytical test device 400 to cleanse the devices. These may include cleaning solutions, disinfectants, and flushing fluids. In various exemplary embodiments, the manifold directs hot water or steam to clean sample, reagents, etc. from the devices. In various exemplary embodiments, the toilet systems using oxygenated water, ozonated water, electrolyzed water, which may be generated on an as-needed basis by the toilet system (this may be internal or external to the toilet).

In various exemplary embodiments, waste from the MFCs is managed based on its characteristics and associated legal requirements. Waste that can be safely disposed is discharged into the sewer line. Waste that can be rendered chemically inert (e.g., heat treatment, vaporization, neutralization) is processed and discharged. Waste that cannot be discharged or treated in the toilet system is stored, and sequestered if necessary, for removal and appropriate handling.

In various exemplary embodiments, the manifold creates sequestered zones for each of these waste categories and ensures that all products are properly handled. In various exemplary embodiments, the manifold directs flushing water and/or cleansing fluids to clean the manifold and MFC. In some embodiments, high-pressure fluids are used for cleaning. In such an embodiment, the high-pressure fluids are not used in the MFC. In some embodiments, the MFC is removed from the backplate interface and all ports are part of the high-pressure cleansing and/or rinse.

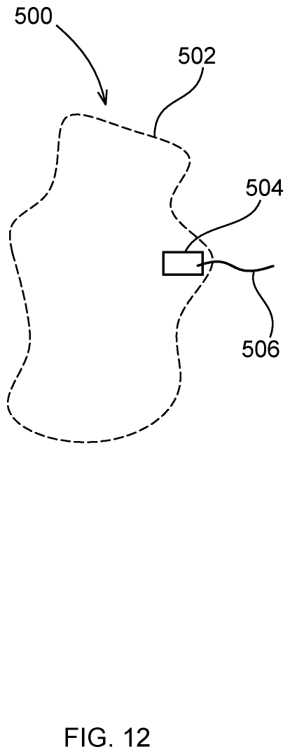

FIG. 12 illustrates a detailed view of an exposure event of excreta to a sensor, according to an embodiment of the disclosure. Exposure event 500 depicts a sample of excreta 502 in contact with sensor 504. Sample 502 is derived from excreta deposited into the toilet. Sample 502 may be in a diluted or undiluted state. Sensor 504 includes a component functionalized to bind with an analyte, such as a biomarker. Alternatively, the component is functionalized to bind with a non-biomarker molecule. Sensor 504 further includes an electrical lead 506 that transmits transduction data to a processor.

Following use of the sensor, the toilet may prepare the sensor for future analysis by removing from the test area waste products and other things that might contaminate the next analysis. This could include flushing the sensor, adding a buffer or stabilizing solution, or adding a gas to remove all liquid from the sensor. There are various options to clean, sanitize, and/or prepare the various components of the involved between uses of the toilet. In one preferred embodiment, hot water is run through the fluidic circuit. In another preferred embodiment, oxygenated water is run through the fluidic circuit. In yet another preferred embodiment, a gas is run through the fluidic circuit to remove any liquid from being in contact with the sensor. Alternatively, cleaning and/or preservation agents are run through the fluid circuit. In still another embodiment, if an analyte receptor, such as an antibody receptor, is used in one or more sensors, the sensors are washed with a solution comprising one or more molecules at a predetermined concentration that can interact with and bind with the receptors in a known and predictive manner. This may be necessary when water or other solvent alone may not be sufficient to displace bound analytes, such as biomarkers, in order to clean the sensor. This cleaning method can act as an indicator to show that the sensors are washed and cleared of analytes before the next subject utilizes the toilet. The analytes may be further cleared from the sensor components using a cleaning or preservation agent dispensed from the toilet.

Additionally, temperature can be critical to the preparation, testing, or post processing of the sensor, the fluidic circuit, or the sample. As such, temperature controls may be included to accommodate those need. The controls could be built into the toilet, built into a fluidic circuit, or a result of adding a reagent to the sample. In one preferred embodiment, a resistive wire acts as a heat source to warm the sample and/or the sensor.

In various exemplary embodiments, the analytical toilet includes additional health and wellness sensors that may be located in a variety of location. In some embodiments, the seat may contain health and wellness sensors to measure pulse, blood pressure, blood oxygenation, electrocardiography, body temperature, body weight, excreta content, excreta weight, excreta volume, excreta temperature, excreta density, excreta flow rate, and other health and wellness indicators. In a preferred embodiment, the seat is attached to the toilet via a powered quick disconnect system that allows the seat to be interchangeable. This facilitates installing custom seats to include user-specific tests based on known health conditions. It also facilitates installing upgraded seats as sensor technology improves.

In various exemplary embodiments, the lid may contain health and wellness sensors that interact with the user's back or that analyze gases in the bowl after the lid is closed.

In various exemplary embodiments, the analytical toilet includes software and hardware controls that are pre-set so that any manufacturer can configure their devices (i.e., analytical test devices) to work in the system. In a preferred embodiment, the system includes a software stack that allows for data channels to transfer data from the sensors in the medical toilet to cloud data systems. The software and hardware controls and/or software stack may be stored in the analytical toilet or remotely. This would allow scientists to place sensors, reagents, etc. in the system to obtain data for their research. It also allows user data to be individually processed, analyzed, and delivered to the user, or their health care provider, digitally (e.g., on a phone, tablet, or computer application). The seat may also contain sensors to measure fluid levels in the toilet. This could include proximity sensors. Alternatively, tubes in fluid communication with the bowl water could be used to determine changes to bowl fluids (e.g., volume, temperature, rate of changes, etc.).

The toilet disclosed herein has many possible uses, including private and public use. Whether for use by one individual, a small group of known users, or general public use, the toilet can detect, monitor, and create one-time and/or trend data for a variety of analytes, such as biomarkers. This data can be used to prompt a user to seek additional medical, health, or wellness advice or treatment; track or monitor a user or population's known condition; and provide early detection or anticipation of a disease or another condition of which a user or population may wish to be aware.

While the present disclosure often notes the sensor and other equipment supporting excreta analysis are located within the toilet, it is possible that some or all of the components are located outside of the toilet. For example, the sample preparation, detection, and processing equipment may be a separate unit adjacent to the toilet which cooperates with the toilet to automatically or semi-automatically receive excreta, prepare a sample of excreta for analysis, test the sample, discard the sample, and prevent cross contamination by cleaning and/or sterilizing portions of the toilet and external equipment that do any portion of the described process.

### EXAMPLE

The following example is provided as part of the disclosure as an embodiment of the present invention. As such, none of the information provided below is to be taken as limiting the scope of the invention.

### Example 1

### Detecting a SARS-COV-2 (COVID-19) Virus

Example 1 is illustrative of a preferred method of detecting a virus. The method comprises:

1) A user releases a sample of excreta into an analytical toilet.

2) A microfluidic system within the analytical toilet directs and transports a sample of the excreta to a sensor. A component of the sensor comprises a FET with a silicon nanowire of approximately 10 nm in diameter that bridges the source and drain electrodes The nanowire is functionalized with the SARS-COV-2 specific antibody ACE2 (ProSci, Inc., Poway, Calif., USA).

3) The sensor detects a change in conductance in the FET due to an interaction event of SARS-COV-2 (COVID-19) virus with the ACE2 antibody bound to the nanowire.

4) The sensor relays the computer-readable data to a processor.

5) The processor processes the data and relays the information to the user or a medical professional at an interface.

6) The user or medical professional takes appropriate action in response to the data.

7) The analytical toilet flushes and cleans the sensors and bowl in preparation for the next user.

The present disclosure relates to a system for detecting body tremors, particularly those symptomatic of an abnormal health or wellness condition for which additional health or wellness care may be desired. The system includes at least two sensors for detecting the weight and/or force exerted by an individual: one which measures weight and/or force on a seat and one which measures weight and/or force on a foot support. The seat sensor is positioned such that it can measure forces a person may exert while seated (e.g. from the rear of the upper legs and adjacent portions of the body). The foot sensor is positioned so it can measure forces from a person's feet and/or lower legs while the person is seated on the seat. The shifting of force, especially weight, back and forth between the seat sensor and the foot sensor can be analyzed to identify a potential tremor. The data with the potential tremor can then be used for a variety of purposes, including comparing the potential tremor data to historical, relevant data; prompting the user to seek additional health or wellness care; reporting the data to a health, wellness, or other care provider; and logging the data for use in future comparisons. Additional sensors can be used to cooperatively detect seat force, cooperatively detect foot force, or additionally detect force applied elsewhere (herein generally referred to with the modifier “tertiary”), such as a backrest, handle, or other structure supporting weight or force, including force not being detected by the seat or foot sensors.

One benefit of the present disclosure is that it allows for identification of potential tremors and can even provide information that can be used by a trained person to help diagnose a tremor as a tremor. It also allows a trained person to monitor tremor activity. A further benefit is that all of this can be implemented for use during a person's normal routine, such as a person's normal restroom routine, and thereby provide regular tracing of tremor or potential tremor activity without having to deviate significantly from their normal routine. The more convenient it is to use the system, the more likely the person is to use it and use it consistently. For example, if implemented into the toilet at a person's home or care facility, the person can simultaneously use the restroom and be monitored for tremors without having to travel to an outside location. Similarly, if implemented with a person's seat, chair, or wheelchair, monitoring can be performed during the person's normal routine. This also can facilitate more frequent monitoring, providing more data with which to assess a person's tremor health. As another example, if implemented at a doctor's office, airport, or laboratory, a person can be monitored for tremors through the familiar activity of sitting down, such as one might do when going to the restroom, waiting in a waiting area, or riding transportation from one location to another, rather than by using an unfamiliar apparatus which requires less familiar activities to complete the monitoring.

Using an unfamiliar system for the monitoring can induce stress in the user and/or increase the chance the user will misuse the apparatus, any of which can negatively affect the quality of the measurement results. Additionally, users may be less likely to use an unfamiliar apparatus, resulting in fewer or no measurements.

As described above, the general application is to use a seat sensor and a foot sensor to detect and measure a portion of force from a user, including weight. After which, the data from those sensors is used to identify potential tremors within the force or weight data, which may then be used for a variety of purposes. There are a variety of ways to implement each of these elements, the selection of which depends on many factors, some of which factors are outside the scope of the invention, such as designer preference, cost, laws, regulations, consumer and stakeholder preferences, and various other market conditions. For example, in one embodiment, the seat sensor is used in conjunction with a toilet seat; in another embodiment, it is used in conjunction with a wheelchair; and in yet another embodiment, the seat sensor is used in conjunction with a more generalized structure that supports force from the rear of the upper legs and/or from the feet (such as a chair on the floor or a restraint system in a space vehicle). Each of these embodiments provides a significant number of acceptable configurations capable of achieving the functional goal of detecting at least a portion of the seat force and generating data based on that force. Similarly, each provides a significant number of acceptable configurations for the implementation of the foot sensor as well as for the analysis and use of the sensor data. Thus, the embodiments disclosed below are a sampling of specific or preferred possible configurations of the elements of the invention and should not be interpreted to limit how the spirit and scope of the invention are achieved.

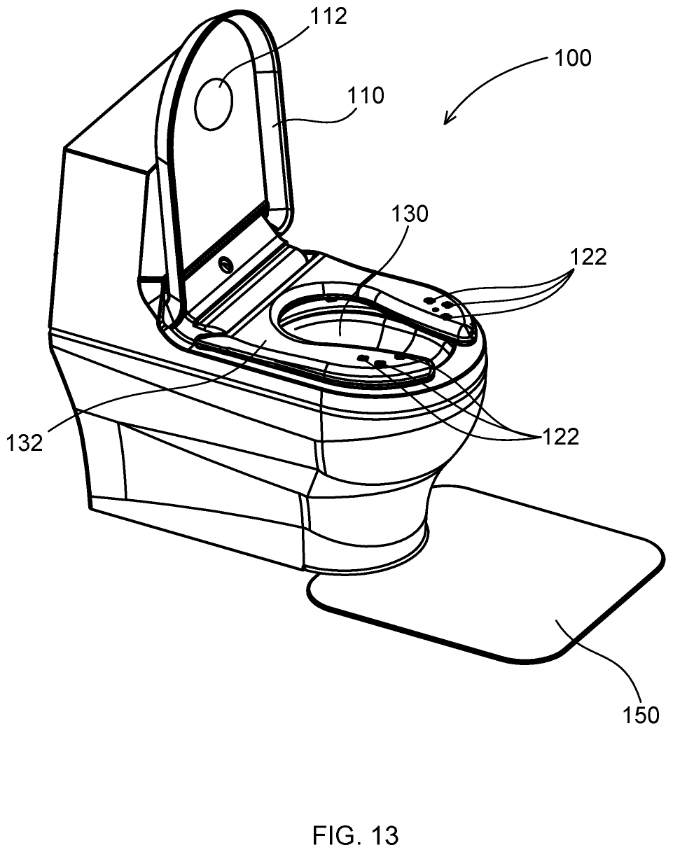

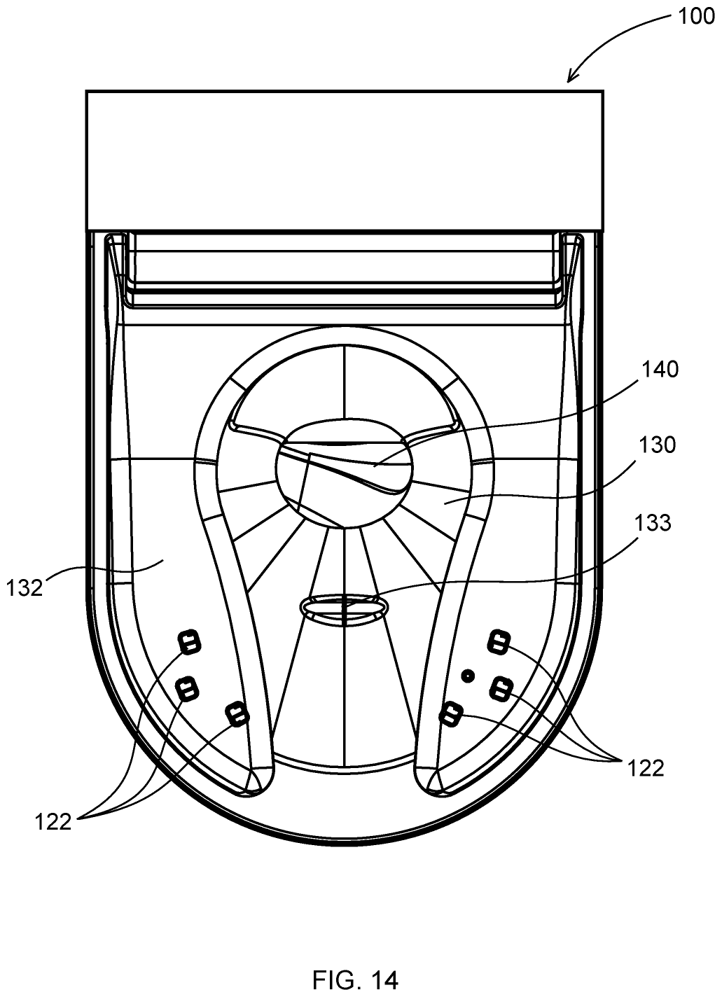

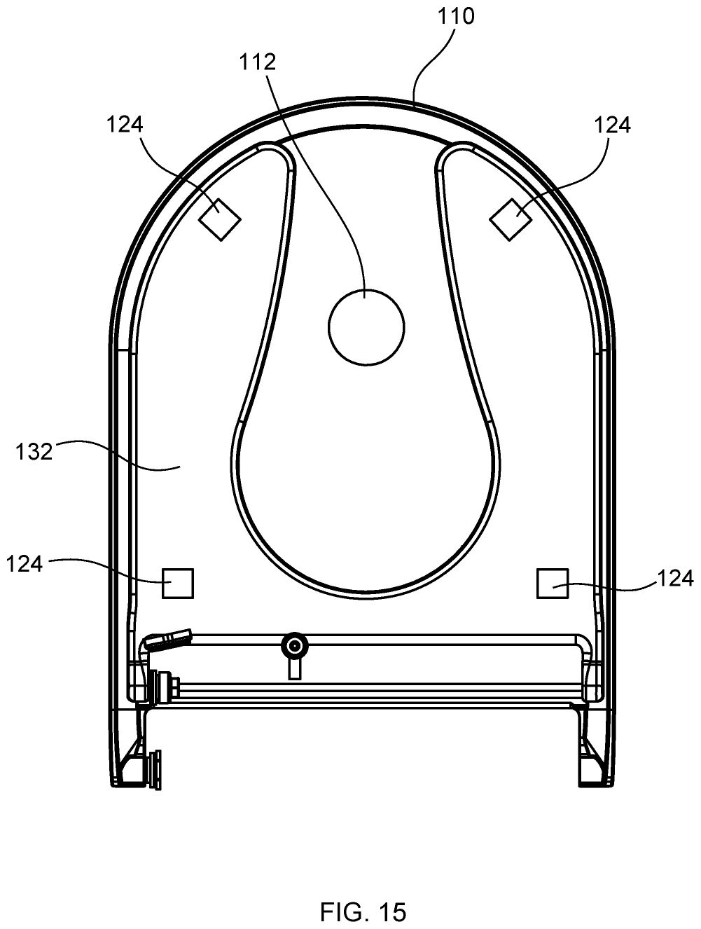

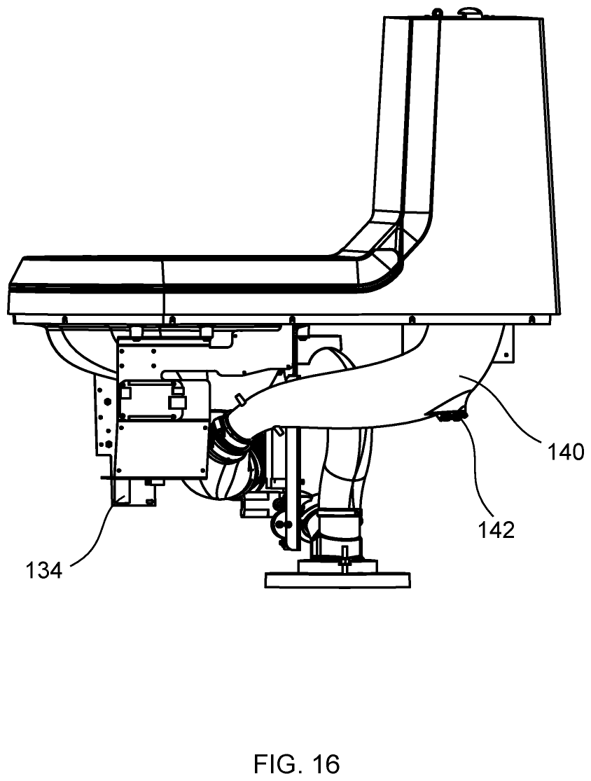

Now referring to FIGS. 13-16, one preferred embodiment of the toilet used in the system is shown. FIG. 13 shows an isometric view of toilet 100 with lid 110 open, showing seat 132 with multiple PPG sensors 122, bowl 130, and foot scale 150. FIG. 14 shows a top view of toilet 100 with lid 110 open, showing seat 132 with multiple PPG sensors 122, bowl 130, and excreta volume measure tube 141. Bowl 130 includes urine slit 133, which captures urine for reading by spectrometer 134. In one embodiment, seat 132 is rotatably attached to toilet 100 in a manner to decrease or minimize the amount of weight transferred from the seat to the hinge and increase or maximize the amount of weight transferred from the seat to toilet 100 away from the hinge; this can facilitate detection and measurement of seat weight by seat force sensors. FIG. 15 is a detail view of the underside of seat 132 with lid 110 behind seat 132. On the underside of seat 132 are seat weight sensors 124. Seat weight sensors 124 are positioned so they transfer force from seat 132 to the surface of toilet 100 directly below seat 132 when it is in the lowered position. Shown on lid 110 is stethoscope 112, which includes a microphone for recording audio sounds from a user's trunk portion of the body, Alternatively, lid 110 has a force sensor, such as an accelerometer, to detect and/or measure the force applied from the user leaning against lid 110, such as when seated on seat 132. FIG. 16 is a detail view showing some of the internal components of toilet 100, including urine volume measure tube 141, urine tube volume sensor 142, and other hardware, which can include a processors for receiving data from the sensors. Alternatively, the processors may be located in the water tank. Alternatively, the processor is remote and the signal from the sensors is transmitted (wired or wirelessly) to the processor.

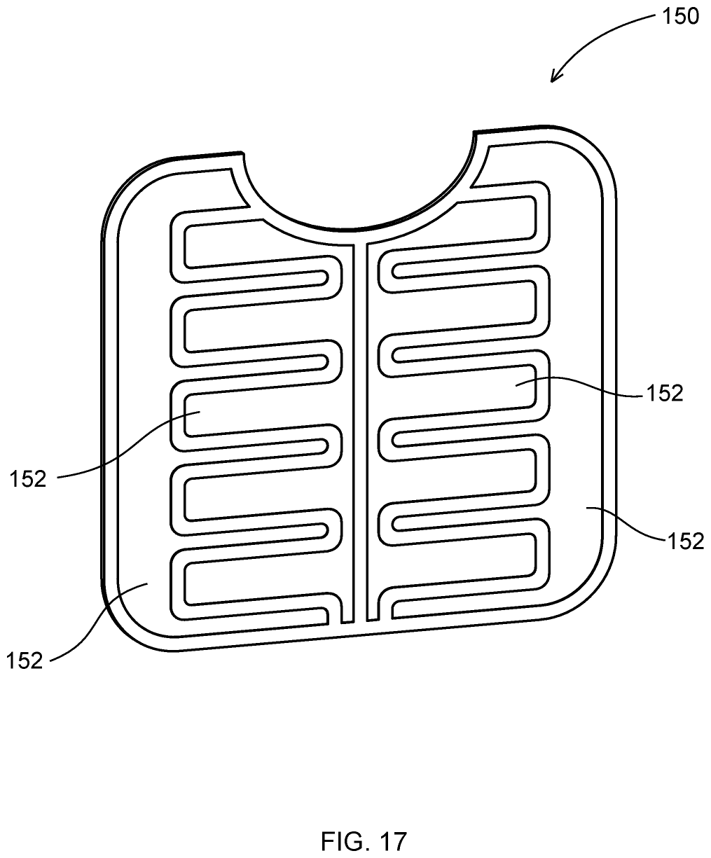

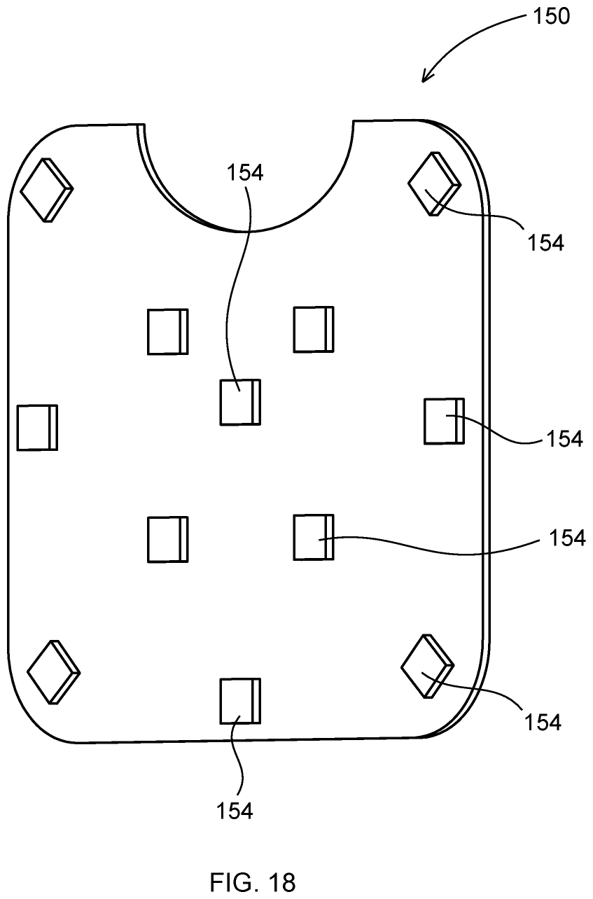

FIGS. 17-18 show one embodiment of a foot pad or foot scale that could accompany a toilet, especially one like that shown in FIGS. 13-16. FIG. 17 shows the top surface of foot scale 150 with bioimpedance sensors 152. To use the bioimpedance sensors, a user would place their bare feet on the top surface, contacting two bioimpedance sensors, allowing the user's bioimpedance to be measured. FIG. 18 shows the bottom of foot scale 150 with multiple foot weight sensors 154 disposed on the bottom surface such that they can transfer force from foot scale 150 to the surface(s) supporting foot weight sensors 154 (e.g., a floor). One side of foot weight sensor 154 attaches to the bottom of the scale and the opposing side of weight sensor 154 contacts the ground. Thus, when a user exerts weight on foot scale 150, the various foot weight sensors 154 can cooperate to determine the weight and/or force the user is exerting on the scale. More preferably, they cooperate to measure changes to the force the user is placing on the scale. Still more preferably, the focus of the measured changes to the force is to identify those which correlate with symptomatic tremors.

In one preferred embodiment, a user walks onto scale 150, and sits down on seat 132, leaving their feet on scale 150. While the user is using the toilet, PPG sensors 122 monitor the user's upper legs; seat weight sensors 124 monitor the portion of the user's weight on seat 132—including minor, apparent fluctuations that are a result of user tremors; foot weight sensors 154 monitor the portion of the user's weight on foot scale 150, and bioimpedance sensors 152 determine the user's bioimpedance. More preferably, the seat weight sensors 124 monitor fluctuations in the person's weight in an order of magnitude and range that correlates with symptomatic tremors.

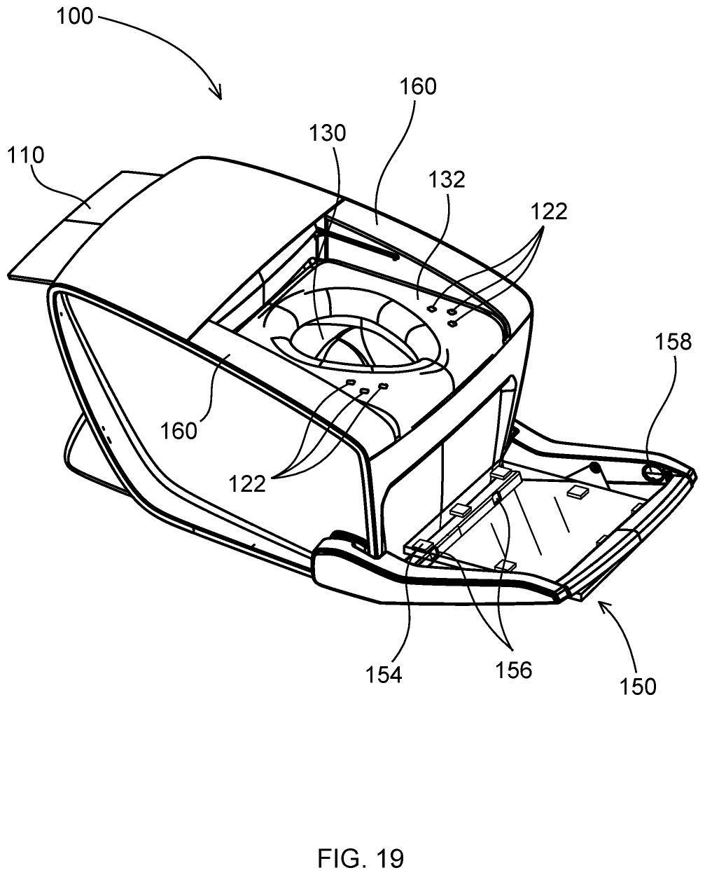

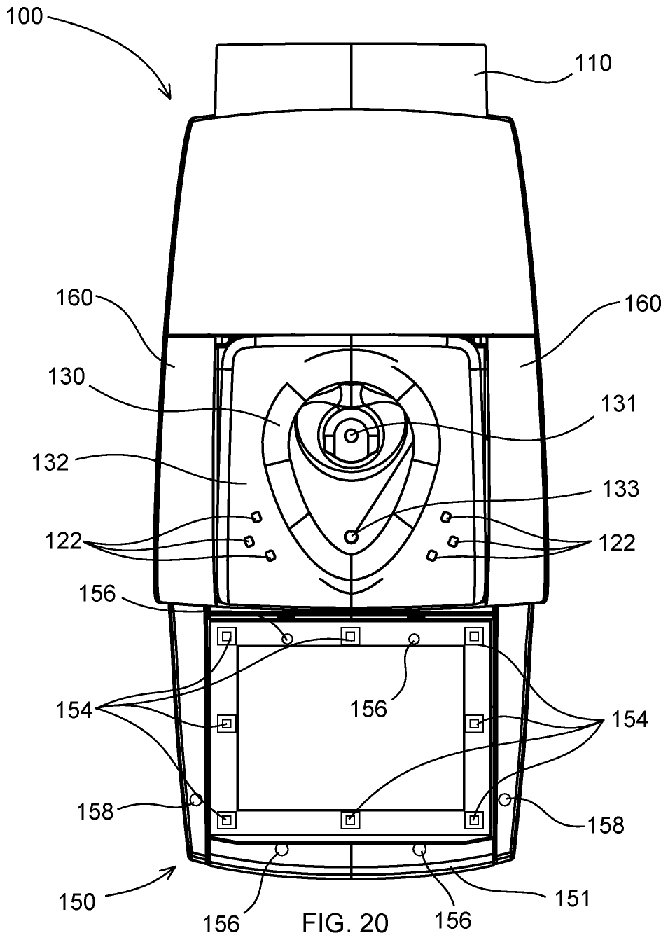

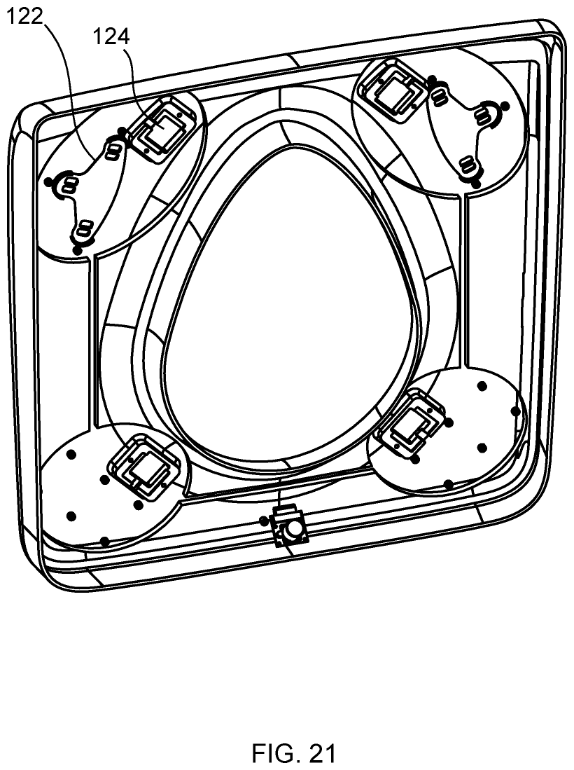

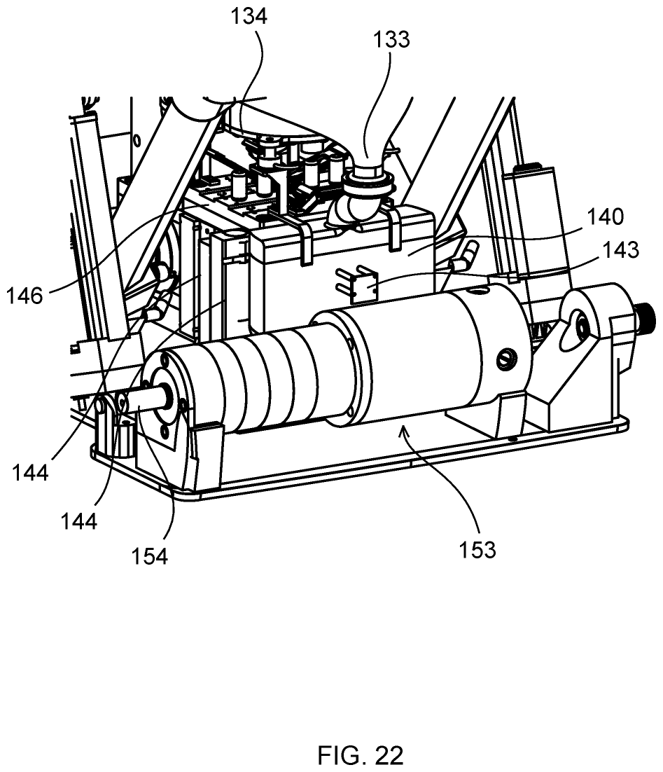

FIGS. 19-22 show another embodiment of the toilet. FIG. 19 shows an isometric view of toilet 100 with lid 110 open, showing seat 132 with multiple PPG sensors 122, bowl 130, foot platform 150, and handles 160. FIG. 20 shows a top view of toilet 100 with lid 110 open, showing seat 132 with multiple PPG sensors 122, bowl 130, foot platform 150, and handles 160. Bowl 130 includes urine receptacle 133 and fecal depository 131. In one preferred embodiment, handles 160 are in a recessed position and can be raised up relative to the toilet. FIG. 21 is a detail view of the underside of seat 132 showing seat weight sensors 124 on the bottom surface of seat 132. FIG. 22 is a detail view showing some of the internal components of toilet 100, including urine receptacle 133, fecal depository 131, urine volume measure chamber 141, urine spectrometer 143, science centers 144, fluid chip receptacle 146, foot platform motor and sensor 152, foot platform motor shaft 153. Foot platform 150 includes frame 151, a glass plate resting on multiple foot weight sensors 154, foot IR imaging sensors 156, and foot near-IR imaging sensors 158. In one preferred embodiment, science centers 144 and fluid chip receptacle 146 are used in conjunction with excreta analysis, including urine samples and emulsified or otherwise processed excreta.

In one preferred embodiment, a user steps onto platform 150, sits down on seat 132, and platform 150 raises via motor 153 so the user's feet easily stay on the glass plate. While the user is using the toilet, PPG sensors monitor the user's upper legs; seat weight sensors 124 monitor the portion of the user's weight on seat 132—including minor, apparent fluctuations that are a result of user tremors; foot weight sensors 154 monitor the portion of the user's weight on foot platform 150, and weight sensor 154 monitors the user's feet and lower legs. More preferably, data from the force sensors are used to monitor for fluctuations in the force of an order of magnitude and range that correlate with symptomatic tremors.

In one preferred embodiment, weight sensor 154 is are able to detect properties of the foot, including foot size and shape, coloring, and subdermal vascular properties. These images can undergo image recognition analysis, the results of which can be compared to preexisting data on the same to generate a report on a user's health. Additionally, data from other sensors can be turned into an image for analysis and report generation. Preferably, the report includes information relative to a user's vascular health. Preferably, the comparison is performed by a neural net which has been trained to recognize commonalities to and differences from preexisting images. When the preexisting images are coupled with known health states and/or conditions of the person from whom the images came, the neural net can suggest correlations between the user's images and health states and/or conditions (including neutral or positive ones). Additionally, when the neural net has examined previous data from the same user, the neural net can compare the user's prior state to his or her current state to report on the relative change. Therefore, it may be useful for user data to be averaged, have the mean taken, used in creating trend data, or otherwise be used in creating a baseline against which to compare new user data as it is generated.

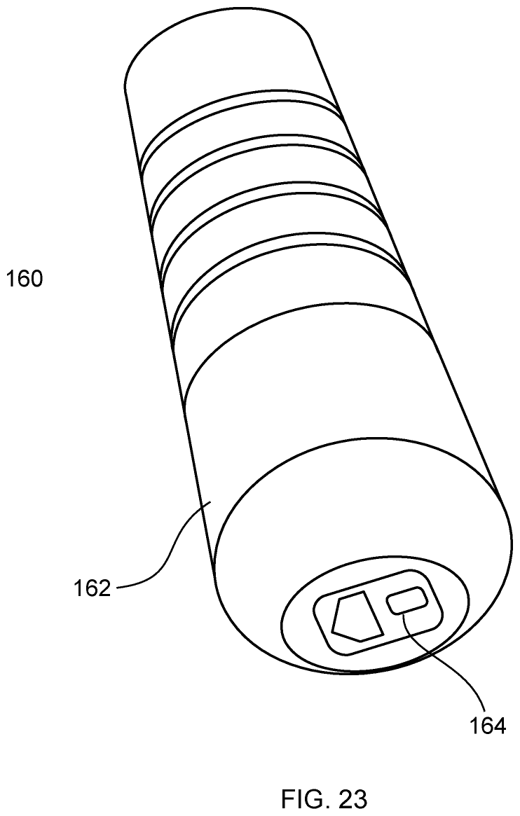

FIG. 23 shows an embodiment of a handle that could accompany a toilet or other apparatus a person may want hand or arm support while using. Handle 160 includes electrical lead 162 and PPG sensor 164. Electrical lead 162 could be a lead for a bioimpedance sensor and/or an ECG sensor. In one preferred embodiment, a handle would be connected to a cord (with wiring) that connects to the toilet. And another preferred embodiment a handle would be mounted to a structure adjacent to the toilet bowl. In either embodiment, a second handle could also be used. Additionally, a handle sensor could detect force from a person, including if the person uses the handle for support while sitting or transitioning between sitting and standing. A second handle may originate from the same connection point to the toilet or a location symmetrically opposite or mirrored from the first handle.

In various exemplary embodiments, the toilet system transmits data recorded by the sensors to a remote storage platform. This data may or may not have been processed prior to transmission. Current data is compared to historical data and/or expected data to determine if there is a deviation. Deviations may be caused by noncompliance with treatment, ineffectiveness of treatment, or with a cause unrelated to the treatment (e.g., elevated body temperature following exercise). The patient and/or healthcare provider is able to access the data and investigate further as appropriate. In various exemplary embodiments, the toilet system may be used to monitor compliance with medical studies and/or the effectiveness of treating symptoms in those studies.

In various exemplary embodiments, urine and feces are separated into different collection basins for independent weighing and/or other analysis. In various exemplary embodiments, cameras estimate the volume of the fecal matter and use the estimated volume to estimate density.

In various exemplary embodiments, the toilet is capable of determining the total weight of excreta and the separate weights of solids and liquids. For example, weight may be measured after completion of an excreta event. Liquids may be allowed to exit the bowl and a new weight measurement taken of the remaining solids. The difference in weight provides the weight of liquid excreta. Alternatively, the urine may be drained off at a known flow rate or through a flow meter to measure urine volume leaving only solids to be weighed.

In various exemplary embodiments, the smart toilet includes at least one sensor that analyzes imaging data. A processor analyzes the data to attempt to compare the user to known users for identification purposes. If a known user is identified, the position of the bowl and/or seat is automatically adjusted to the preferred position of the user. If the user is unknown or does not have a record preferred position, the processor analyzes the physical characteristics of the user (e.g., height, waist height, length of upper and lower legs) and adjusts the position of the bowl and/or seat accordingly. In some embodiments, facial recognition is used to identify users.

In various preferred embodiments, the system may identify a user based on their face, hand, or foot. In various preferred embodiments, the sensor may include a CCD (charge-coupled device) or MOS (metal oxide semiconductor), including CMOS (complementary metal oxide semiconductor). The sensor can be used, with proper calibration such as taking the data at a known distance, to measure the length of major bones. This data can then be used to customize the toiler position for an unknown user.

In accordance with the present disclosure, a smart toilet that includes mechanical, hydraulic, power, and data connections to accommodate a combination of health measuring tools is provided. An electrical connection to provide power to the health measuring tools is also provided.

In various exemplary embodiments, the toilet system includes a variety of sensors that allow it to make measurements related to the health and wellness of a user. The sensors may include, but are not limited to, CCD, MOS, CMOS, spectrometer, chromatograph, FET, nano-FET, MOSFET, mass spectrometer, electrode, microphone, load cell, pressure gauge, PPG, electrocardiograph, ultrasound, thermometer, rheometer, durometer, pH detector, or scent detector.

In various exemplary embodiments, some toilet system sensors interact with excreta to measure a characteristic of the excreta. The characteristics measured may include, but are not limited to, comprises volume, flow rate, color, weight, density, content, temperature, clarity, pH, settled gradient, or flow geometry of urine and weight, color, consistency, volume, density, content, temperature, pH, size and shape, excretion profile, sounds, or gas or fumes of feces.

In various exemplary embodiments, some sensors interact with the user to measure a health and wellness characteristic. The characteristics may include, but are not limited to, electrocardiogram, pulse, blood pressure, blood oxygenation, hypoxia, heart rhythm, respiration, body temperature, body tremors, hydration, or electrolyte balance.

In various exemplary embodiments, the toilet system includes sensors for measuring the user's weight. This includes separate sensors for determining user weight on the seat and user weight on the user's feet. The user's weight that is supported by their feet is preferably measured using a platform adjacent to the toilet on which the user's feet are placed while sitting and while being lowered or raised from the toilet.

In the preferred embodiments, the toilet also includes health assessment devices supported by the frame. Examples of such devices include imaging cameras, flow spectrometers, volume measurement devices, body weight sensors, and gas analyzers. Toilets with such devices are described in the patents and published applications cited in the Background section above.

All patents, published patent applications, and other publications referred to herein are incorporated herein by reference. The invention has been described with reference to various specific and preferred embodiments and techniques. Nevertheless, it is understood that many variations and modifications may be made while remaining within the spirit and scope of the claims.

What is claimed is:

1. An analytical toilet comprising:
a bowl for receiving excreta from a user;
a seat for the user; and
a sensor for detecting a symptom of the user;
wherein the sensor transmits data to a processor that uses the data to measure a symptom of the user; and
and the processor uses the symptom to track user compliance with medication or treatment regimen.

2. The analytical toilet of claim 1 wherein the symptom is a characteristic of the user's excreta.

3. The analytical toilet of claim 2 wherein the characteristic comprises volume, flow rate, color, weight, density, content, temperature, clarity, pH, settled gradient, or flow geometry of urine.

4. The analytical toilet of claim 2 wherein the characteristic comprises weight, color, consistency, volume, density, content, temperature, pH, size and shape, excretion profile, sounds, or gas or fumes of feces.

5. The analytical toilet of claim 2 wherein the characteristic is the level of an analyte in the excreta.

6. The analytical toilet of claim 1 wherein the symptom is a characteristic of the user's cardiovascular system.

7. The analytical toilet of claim 6 wherein the characteristic comprises electrocardiogram, pulse, blood pressure, blood oxygenation, hypoxia, or heart rhythm.

8. The analytical toilet of claim 1 wherein the symptom is a characteristic of the user's physical condition.

9. The analytical toilet of claim 8 wherein the characteristic comprises respiration, body temperature, body tremors, hydration, or electrolyte balance.

10. The analytical toilet of claim 1 wherein the sensor comprises CCD, MOS, CMOS, spectrometer, chromatograph, FET, nano-FET, MOSFET, mass spectrometer, electrode, microphone, load cell, pressure gauge, PPG, electrocardiograph, ultrasound, thermometer, rheometer, durometer, pH detector, or scent detector.

11. A method for monitoring a patient comprising:
providing an analytical toilet comprising:
a bowl for receiving excreta from a user;
a seat for the user; and
a sensor for detecting a symptom of the user;
having a user use the toilet to deposit excreta;
detecting a symptom of the user with the sensor;
transmitting data from the sensor to a processor; and
using the data to measure the symptom and compare it to the user's normal or expected symptoms; and
using the comparison to determine user compliance with medication or treatment regimen.

12. The method of claim 11 wherein the symptom is a characteristic of the user's excreta.

13. The method of claim 12 wherein the characteristic comprises volume, flow rate, color, weight, density, content, temperature, clarity, pH, settled gradient, or flow geometry of urine.

14. The method of claim 12 wherein the characteristic comprises weight, color, consistency, volume, density, content, temperature, pH, size and shape, excretion profile, sounds, or gas or fumes of feces.

15. The method of claim 12 wherein the characteristic is the level of an analyte in the excreta.

16. The method of claim 11 wherein the symptom is a characteristic of the user's cardiovascular system.

17. The method of claim 16 wherein the characteristic comprises electrocardiogram, pulse, blood pressure, blood oxygenation, hypoxia, or heart rhythm.

18. The method of claim 11 wherein the symptom is a characteristic of the user's physical condition.

19. The method of claim 18 wherein the characteristic comprises respiration, body temperature, body tremors, hydration, or electrolyte balance.

20. The method of claim 11 wherein the sensor comprises CCD, MOS, CMOS, spectrometer, chromatographs, FET, nano-FET, MOSFET, mass spectrometer, electrode, microphone, load cell, pressure gauge, PPG, electrocardiograph, ultrasound, thermometer, rheometer, durometer, pH detector, or scent detector.
